(19) United States
(12) Patent Application Publication
(10) Pub. No.: US 2025/0208246 A1
(43) Pub. Date: Jun. 26, 2025

(54) SYSTEM, METHOD AND COMPUTER-ACCESSIBLE MEDIUM FOR DETERMINING ROTATIONAL INVARIANTS OF CUMULANT EXPANSION FROM ONE OR MORE ACQUISITIONS WHICH CAN BE MINIMAL

(71) Applicant: NEW YORK UNIVERSITY, New York, NY (US)

(72) Inventors: Santiago COELHO, New York, NY (US); Els Fieremans, New York, NY (US); Dmitry S. Novikov, New York, NY (US)

(21) Appl. No.: 18/923,344

(22) Filed: Oct. 22, 2024

Related U.S. Application Data
(63) Continuation of application No. PCT/US2023/019622, filed on Apr. 24, 2023.
(60) Provisional application No. 63/333,856, filed on Apr. 22, 2022.

(51) Int. Cl. G01R 33/56; G01R 33/563

(52) U.S. Cl. CPC G01R 33/5608; G01R 33/56341

(57) ABSTRACT

Exemplary system, method and computer arrangement for determining invariants associated with at least one physical structure are described, which can include a receipt of at least one particular component which is a component of a diffusion tensor and/or a component of a covariance tensor, whereas the at least particular component is associated with the at least one physical structure. Then, it is possible to generate the invariants of the diffusion tensor and/or the covariance tensor based on the particular component. The physical structure can be (i) a biological tissue, (ii) a composite material, (iii) a continuous medium, and/or (iv) a random medium. For the biological tissue, the particular component can be based on diffusion magnetic resonance (dMR) image of the tissue.

### CROSS-REFERENCE TO RELATED APPLICATION(S)

This application relates to and claims priority from International Application no. PCT/US2023/019622 filed on Apr. 24, 2023 and relates to and claims priority from U.S. Patent Application No. 63/333,856, filed on Apr. 22, 2022, the entire disclosure of which is incorporated herein by reference.

### STATEMENT REGARDING FEDERALLY SPONSORED RESEARCH

This invention was made with government support under Grant Nos. R01 NS088040 (NINDS), R01 EB027075 (NIBIB), awarded by the NIH, and the Center of Advanced Imaging Innovation and Research, a NIBIB Biomedical Technology Resource Center: P41 EB017183. The government has certain rights in the invention.

### BACKGROUND INFORMATION

Diffusion magnetic resonance imaging (dMRI), based on diffusion nuclear magnetic resonance (NMR), is a non-invasive imaging modality that provides information about the architecture of any physical structure, in which the spin-carrying atoms or molecules can diffuse over a certain time set by the NMR measurement. Such physical structures can include, but are not limited to, composite materials, continuous media, random media, porous media, porous rocks, and biological tissues. For example, biological tissue architecture restrics the random motion of water molecules, which makes dMRI of water protons an essential part of any clinical or research brain MRI protocol. Typical experimental settings probe such motion at a scale of micrometers or tens of micrometers, orders of magnitude below MRI imaging resolution (see, e.g., Kiselev, 2017; Novikov et al., 2019; and Alexander et al., 2019). Hence, tissue microstructure imaging with dMRI can become sensitive, and possibly specific, to developmental, aging and disease processes that originate at this scale and could provide biomarkers of said processes (see, e.g., Assaf, 2008; and Jelescu & Budde, 2017). Herein, both diffusion NMR and diffusion MRI can be referred to as dMRI, given that diffusion NMR can be seen as a particular case of dMRI when imaging is not performed, but diffusion encoding methodology is similar for the purposes of the present disclosure.

The information content of the dMRI signal depends on the level of coarse-graining (see, e.g., Novikov et al., 2019) over the diffusion length controlled by the diffusion time t. With sufficiently long t approximately 30-50 ms or greater, typically used in the clinic, each tissue compartment (e.g., intra-axonal space, extra-axonal space within each fiber fascicle, etc) can be asymptotically considered as fully coarse-grained, such that diffusion in it becomes approximately Gaussian. Assuming Gaussian diffusion in each compartment drastically simplifies biophysical modeling. Indeed, the signal S^{α}exp(−tr B D^{α}) (e.g., normalized to 1 in the absence of diffusion weighting) from any “Gaussian compartment” labeled by α and defined by its diffusion tensor D^{α}, becomes fully encoded by a 3×3 symmetric B-tensor (see, e.g., Westin et al., 2016; and Topgaard, 2017). The overall signal at clinically-relevant moderate diffusion weightings is then represented by the cumulant expansion (van Kampen, 1981; Kiselev, 2010).

$$ln S = - {B}_{ij} {D}_{ij} + \frac{1}{2} {B}_{ij} {B}_{kl} {C}_{ijkl} + 𝒪 ( {b}^{3} ) , \qquad ( 1 )$$

where b=tr B is the dMRI b-value, see Materials and Methods for details. Here, Einstein's convention of summation over repeated Roman indices is assumed hereon, and

$${D}_{ij} = 〈 〈 {D}_{ij}^{α} 〉 〉 = 〈 {D}_{ij}^{α} 〉 = {\sum }_{α} {f}_{α} {D}_{ij}^{α} , \qquad ( 2 )$$

$${C}_{ijkl} = 〈 〈 {D}_{ij}^{α} {D}_{kl}^{α} 〉 〉 = 〈 {D}_{ij}^{α} {D}_{kl}^{α} 〉 - 〈 {D}_{ij}^{α} 〉 〈 {D}_{kl}^{α} 〉 ,$$

are components of the overall diffusion and covariance tensors D and C. Tensor D has 6 independent parameters, or “degrees of freedom” (dof), and C has 21 dof (see FIGS. 1a-1e). Angular brackets [symbol] . . . [symbol] denote averages over the diffusion tensor distribution [symbol] (D) that characterizes the tissue—or, equivalently, over the compartment index α, and double brackets [symbol] . . . [symbol] denote the cumulants (see, e.g., van Kampen, 1981).

For a model-independent signal representation (1), which can be utilized even without assuming Gaussian diffusion in all compartments, a fundamental problem is to classify symmetries and define tensor invariants, i.e., combinations of tensor components that are independent of the choice of basis (such as, but not limited to, mean diffusivity). These rotational invariants facilitate the generation of basis-independent scalar maps. They can be the easiest to visualize (as opposed to multi-dimensional objects such as tensors), and to study as candidate markers of development, aging and disease.

Tensor invariants can serve as ideal hardware-independent “fingerprint” of a dMRI signal; their information content can underpin future classifiers of development, pathology and aging, for any tissue or organ, such as, but not limited to, brain. A practical problem is to relate the invariants to tissue properties, and to find ways of their fast and robust estimation.

Thus, it may be beneficial to provide exemplary systems, methods and computer-accessible medium can overcome at least some of the deficiencies described herein above.

### SUMMARY OF EXEMPLARY EMBODIMENTS

To that end, it is possible to provide exemplary systems, methods and computer-accessible medium according to exemplary embodiments of the present disclosure, which can be referred to herein, as an example only, as exemplary systems, methods and computer-accessible medium utilizing and/or providing Rotational Invariants of the Cumulant Expansion (or “RICE” techniques/procedures, methods, systems and/or computer-accessible medium).

In these exemplary embodiments of the present disclosure, a full classification of rotational invariants of the cumulant expansion (RICE), i.e., invariants of diffusion tensor D and covariance tensor C (in total, 3+18 RICE invariants) can be provided, in terms of irreducible representations of the group of rotations, their geometric meaning can be elucidated, and the invariants can be related to the problem of addition of quantum-mechanical angular momenta.

In additional exemplary embodiments of the present disclosure, exemplary formulas can be provided and utilized for calculating of most or all RICE invariants, and connect them with tissue biophysics embodied by the distribution [symbol] (D). It is possible to express conventional scalar contrasts—mean diffusivity (MD), fractional anisotropy (FA), mean kurtosis (MK), and microscopic fractional anisotropy (μFA)—in terms of some of the RICE invariants.

According to certain exemplary embodiments of the present disclosure, iRICE acquisitions can be provided based on the icosahedral directions with the smallest number of measurements required to determine, e.g., only the tensor elements that yield the above conventional contrasts in 1-2 minutes for the whole brain on a clinical scanner.

With the advent of precision medicine and quantitative imaging, tensor invariants can provide a parsimonious and hardware-independent “fingerprint” of a dMRI signal. Representing the signal's information content in terms of scalar invariant maps according to the underlying symmetries can improve and moreover underpin machine learning classifiers of brain pathology, development and aging, while fast iRICE protocols can enable translation of advanced dMRI into a clinical practice.

According to further exemplary embodiments of the present disclosure, exemplary systems, methods and computer-accessible medium can be provided for determining invariants associated with at least one physical structure. Using such exemplary embodiments, it is possible to receive at least one particular component which is a component of a diffusion tensor and/or a component of a covariance tensor. The particular component(s) can be associated with the physical structure(s). In such exemplary embodiments, it is also possible to generate the invariants of the diffusion tensor and/or the covariance tensor based on the particular component. For example, the physical structure can be, but not limited to, (i) a biological tissue, (ii) a composite material, (iii) a continuous medium, and/or (iv) a random medium, (v) porous media and/or (vi) porous rocks. The particular component can be based on diffusion magnetic resonance (dMR) image of the physical structure (which can be, but not limited to, at least one tissue). The invariants can associated with at least one parameters of such structure (e.g., the tissue).

In certain exemplary embodiments of the present disclosure, the diffusion tensor can be split into a scalar part of degree 0 and a symmetric trace-free (STF) part of degree 2. Alternatively or in addition, the covariance tensor can be split into a fully symmetric part and an asymmetric part. Alternatively or in addition, the fully symmetric part of the covariance tensor can be split into a first part of degree 0, a second part of degree 2, and a third part of degree 4. Alternatively or in addition, the asymmetric part can be split into a first part of degree 0 and a second part of degree 2.

According to additional exemplary embodiments of the present disclosure, the first parts of the diffusion tensor, of the fully symmetric part of the covariance tensor, and of the asymmetric part of the covariance tensor, respectively, can be used to generate of the invariants which can be proportional to full traces thereof. Alternatively or in addition, the second part of the diffusion tensor can be used to generate at least two of the invariants (e.g., intrinsic variants) which can be based on traces of second and third powers of such second part. Alternatively or in addition, the second part of the fully symmetric part of the covariance tensor can be used to generate at least two of the invariants which are based on traces of second and third powers of such second part. Alternatively or in addition, the second part of the asymmetric part of the covariance tensor can be used to generate at least two of the invariants which can be based on traces of second and third powers of such second part. Alternatively or in addition, the third part of the fully symmetric part of the covariance tensor can be used to generate at least four of the invariants which can be based on traces of second, third, fourth and fifth powers of such third part. Alternatively or in addition, the third part of the fully symmetric part of the covariance tensor can be used to generate at least two invariants based on traces of cubic powers of at least two eigentensors, that are determined from an eigentensor decomposition of such third part.

In yet additional exemplary embodiments of the present disclosure, eigenbases of the second part of the fully symmetric part of the covariance tensor, the second part of the asymmetric part of the covariance tensor, and the third part of the fully symmetric part of the covariance tensor can be used to generate at least one of the invariants of the covariance tensor, based on relative orientations of the eigenbases. Further, a first set of the invariants of the covariance tensor can be given by parameters of a rotation of the eigenbasis of the second part of the asymmetric part of the covariance tensor relative to the eigenbasis of the second part of the fully symmetric part of the covariance tensor. Alternatively or in addition, a second set of the invariants of covariance tensor can be given by parameters of a rotation of the eigenbasis of the third part of the fully symmetric part of the covariance tensor relative to the eigenbasis of the second part of the fully symmetric part of the covariance tensor. The eigenbasis of the third part of the fully symmetric part of the covariance tensor can be generated based on the eigenbasis corresponding to a largest eigenvalue of an eigentensor decomposition thereof.

According to further exemplary embodiments of the present disclosure, a kurtosis tensor can be generated based on the fully symmetric part of the covariance tensor, and kurtosis invariants can be generated based on the invariants of the fully symmetric part of the covariance tensor. Alternatively or in addition, it is possible to utilize the invariants to determine contrasts, which includes (i) mean, axial or radial diffusivity, (ii) fractional anisotropy, (iii) mean, axial and/or radial kurtosis, and/or (iv) microscopic fractional anisotropy. Alternatively or in addition, it is possible to utilize the invariants to determine contrasts which includes (i) isotropic variance, and/or (ii) anisotropic variance.

In yet further exemplary embodiment of the present disclosure, it is possible to generate compartmental tensor covariances associated with the tissue parameters. The compartmental tensor covariances can include size-size covariance, shape-shape covariance, and size-shape covariance. It is additionally possible to generate a size-shape correlation.

According to further exemplary embodiments of the present disclosure, it is possible to utilize parameter maps (e.g., RICE maps) as inputs to machine-learning or artificial-intelligence classifiers, such as, but not limited to, neural networks, trained for an automatic pathology detection and staging. One of the advantages of such parameter maps as inputs for machine-learning classifiers can be in their independence of the hardware and measurement protocol. In such exemplary manner, it is possible to combine training data from multiple sites, scanner manufacturers, field strengths, and other differing tissue-independent characteristics, that could otherwise limit the effectiveness of training of such classifiers.

According to certain further exemplary embodiments of the present disclosure, exemplary systems, methods and computer-accessible medium can be provided for determining invariants associated with at least one physical structure. Using such exemplary embodiments, it is possible to receive information related to the at least one diffusion magnetic resonance (dMR) image of the at least one physical structure. It is then possible to generate the invariants of a diffusion tensor and/or covariance tensors using (i) a particular number of acquisitions and/or particular directions, and (ii) the information.

For example, in an exemplary embodiments, the particular number and the particular directions of the diffusion acquisitions can correspond to spherical designs. The spherical designs can correspond to at least one number of directions that can be configured to form a spherical design. The spherical designs can be provided by half of the octahedron vertices, half of icosahedron vertices, all octahedron vertices, or all icosahedron vertices. It is also possible to generate the mean diffusivity and the mean kurtosis based on an un-weighed diffusion image, 6 icosahedron vertices for a first b-shell, and/or 6 icosahedron vertices for a second b-shell. The first b-shell can be selectable as approximately b=1 ms/m\hat{ }2, and the second b-shell can be selectable as approximately b=2 ms/m\hat{ }2.

In yet another exemplary embodiment of the present disclosure, it is possible to generate the mean diffusivity, fractional anisotropy, the mean kurtosis, and/or microscopic fractional anisotropy based on an un-weighed diffusion image, 6 icosahedron vertices for the first b-shell, 6 icosahedron vertices for the second b-shell, and/or a spherical tensor encoding (STE) acquisition at the third b-value. The first b-shell can be selectable as approximately b=1 ms/m\hat{ }2. The second b-shell can be selectable as approximately b=2 ms/m\hat{ }2. The third b-value (STE) can be selectable as approximately b=1.5 ms/m\hat{ }2.

According to certain further exemplary embodiments of the present disclosure, exemplary systems, methods and computer-accessible medium can be provided for determining at least one component of at least one tensor associated with at least one physical structure. Using such exemplary embodiments, it is possible to receive first information related to at least one diffusion magnetic resonance (dMR) image of the physical structure(s), receive second information related to at least one constraint on the component(s) of at least one tensor. It is then possible to generating the component(s) which is/are a component of a diffusion tensor and/or a component of a covariance tensor based on the first information and the second information.

In a further exemplary embodiment of the present disclosure, the constraint(s) can be for the value of a part of the tensor(s) of degree 4. The value of the part of the tensor(s) of degree 4 can be zero. The component(s) of the covariance tensor can be estimated in a symmetric trace-free (STF) basis. Alternatively or in addition, the constraint(s) can be formulated for the STF basis coefficients of degree 4. Alternatively or in addition, the constraint(s) can be on a further part of the tensor(s) of degree 2 to be axially symmetric. Alternatively or in addition, the constraint(s) can be on the part and the further part of the tensor(s) can be aligned therebetween or with an eigenbasis of the diffusion tensor.

These and other objects, features and advantages of the exemplary embodiments of the present disclosure will become apparent upon reading the following detailed description of the exemplary embodiments of the present disclosure, when taken in conjunction with the accompanying claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is a schematic diagram of an exemplary tensor decomposition according to certain exemplary embodiments of the present disclosure;

FIG. 1B is a diagram of a summary of typical tensor encodings and their access to the parts of the previous decomposition;

FIG. 1C is an illustration of an axially symmetric B-tensor parametrization;

FIG. 1D is a diagram of the decomposition of compartmental diffusion tensors D^{α} in STF/SH basis;

FIG. 1E is an illustration of all size and shape covariances present in the C-tensor.

FIG. 1F is a diagram of examples of exemplary spherical designs which can be used for fast protocols according to exemplary embodiments of the present disclosure;

FIG. 2A shows the irreducible decompositions of C and D tensors, Eqs. (10) and (11), for a white matter voxel of the corpus callosum—highly aligned fibers—Glyphs are rescaled to a similar size and color-coded by the sign (red=positive), while radius represents the absolute value.

FIG. 2B is an illustration of set of irreducible decompositions of C- and D-tensors for a white matter voxel of the longitudinal superior fasciculus—crossing fibers—according to exemplary embodiments of the present disclosure;

FIG. 2C is an illustration of a set of representations of the eigentensor decomposition for the crossing fiber voxel shown in FIG. 2b in accordance with the exemplary embodiments of the present disclosure;

FIG. 3 is an illustration of the full irreducible decomposition of C- and D-tensors in STF basis according to the exemplary embodiments of the present disclosure;

FIG. 4 is an illustration of various exemplary RICE maps for a normal brain of a 33 year old male;

FIG. 5A is a set of maps and a graph of the ‘energy’ distribution of D, W and A tensors for each degree e and their histograms for white and gray matter voxels according to exemplary embodiments of the present disclosure;

FIG. 5B is a set of maps and a graph proportions of ‘energy’ distribution along the main axis each voxel relative to the total value at each degree according to exemplary embodiments of the present disclosure;

FIG. 6A is a set of maps and a graph parallel and perpendicular projections of the diffusion tensor onto the principal fiber basis according to exemplary embodiments of the present disclosure;

FIG. 6B is a set of maps and a graph parallel and perpendicular projections of the kurtosis tensor onto the principal fiber basis according to exemplary embodiments of the present disclosure;

FIG. 7A is a set of maps providing an MD, FA, and MK comparison of iRICE maps (top) versus fully sampled RICE maps (bottom) for a healthy volunteer according to exemplary embodiments of the present disclosure;

FIG. 7B is a set of scatter plots which include whole brain voxels from 3 normal volunteers comparing MD, FA, and MK from iRICE versus fully sampled RICE maps according to the exemplary embodiments of the present disclosure;

FIG. 8A is a set of maps providing an MD, FA, MK, and μFA comparison of iRICE maps (top) versus fully sampled RICE maps (bottom) for a healthy volunteer according to the exemplary embodiments of the present disclosure;

FIG. 8B is a set of scatter plots which include whole brain voxels from 3 normal volunteers comparing MD, FA, MK, and μFA from iRICE vs fully sampled RICE maps according to the exemplary embodiments of the present disclosure;

FIG. 9 is a set of exemplary flowcharts of methods which can include exemplary actions which can be taken to determine exemplary tissue parameters according to exemplary embodiments of the present disclosure; and

FIG. 10 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

### Exemplary Probing of C Tensor

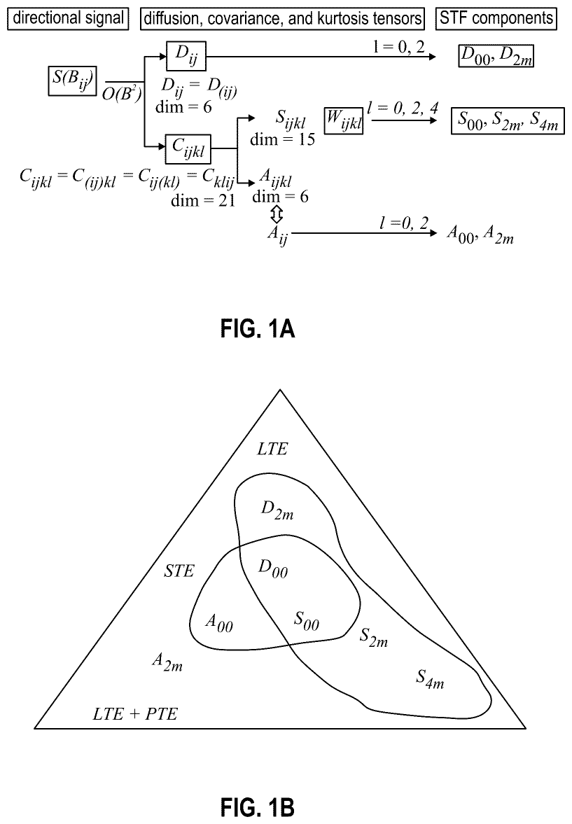

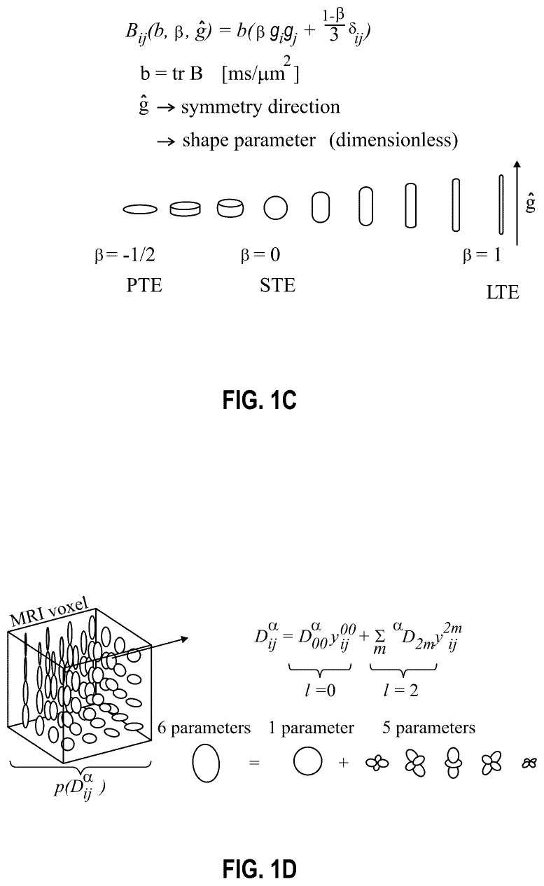

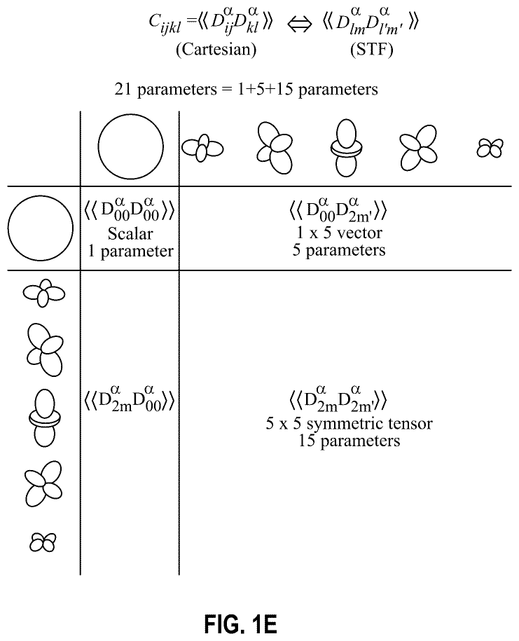

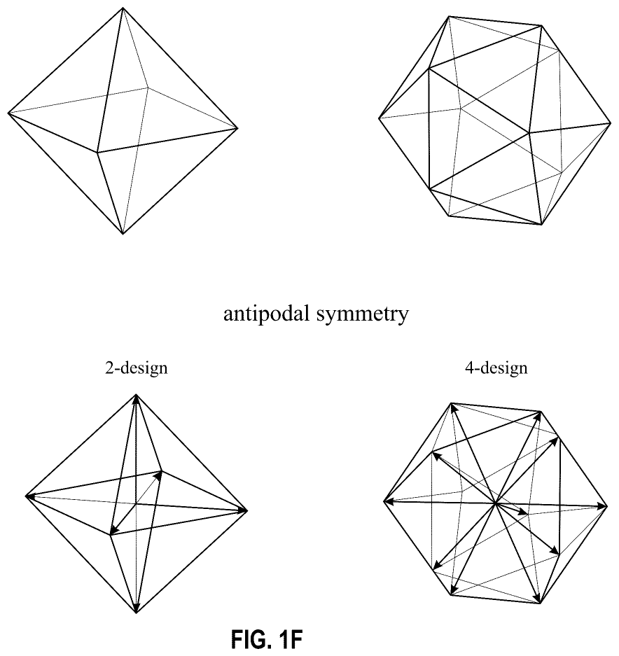

To obtain all 21 dof of C, the necessary (but not sufficient) condition can be rank B>1 (see, e.g., Westin et al., 2016). This should be contrasted with diffusion kurtosis imaging (DKI) (see Jensen et al., 2005) yielding only S, the fully symmetric part of C (see, e.g., FIGS. 1a-1f), proportional to the kurtosis tensor

$${W}_{ijkl} = \frac{3}{{\overline{D}}^{2}} {S}_{ijkl} , {S}_{ijkl} = {C}_{( ijkl )} \qquad ( 3 )$$

with 15 dof, as it involves only linear tensor encoding (LTE, rank B=1) provided by PGSE (see, e.g., Stejskal and Tanner, 1965). In this exemplary case, D̅=Dᵢᵢ/3 is mean diffusivity, and symmetrization over tensor indices between ( . . . ) is assumed henceforth (such as

$${T}_{( ij )} = \frac{1}{2 !} ( {T}_{ij} + {T}_{ji} ) ,$$

and similarly for all n! permutations of indices for n-th order tensors (see, e.g., Thorne, 1980).) Estimation of higher-order cumulants (see, e.g., Ning et al., 2021) is challenging due to a small convergence radius of the cumulant series (see, e.g., Kiselev & Il'yasov, 2007).

The number of nonzero eigenvalues of the B tensor generally reflects how many dimensions of the diffusion process are being probed simultaneously. The requirement rank B>1 for probing the full C tensor can mean that some of its dof are describing diffusion along more than one dimension. According to the exemplary embodiments of the present disclosure, without the loss of generality, we focus on axially symmetric B (see, e.g., FIG. 1c), in components:

$${B}_{ij} ( b , β , \hat{g} ) = b ( β {g}_{i} {g}_{j} + ( 1 - β ) / 3 {δ}_{ij} ) , \qquad ( 4 )$$

parametrized by its trace b giving the overall scale; the unit vector ĝ along its symmetry axis; and the dimensionless shape parameter β (see, e.g., Eriksson et al., 2015). Compared to conventional linear tensor encoding (LTE, β=1), according to the exemplary embodiments of the present disclosure, varying the extra degree of freedom β changes the B-tensor shape, e.g., β=0 for spherical encoding (STE, isotropic B-tensor), and β=−1/2 for planar encoding (PTE, two equal nonzero eigenvalues) (see, e.g., Cory et al., 1990; Mitra, 1995; Mori & Van Zijl, 1995; Shemesh et al., 2015). Non-axially symmetric B-tensors are not necessary for accessing all [symbol] (b²) information, and are typically not employed.

### Exemplary Irreducible Decomposition of the C Tensor

The complementary approach based on representations of the SO(3) group of rotations (see, e.g., Backus, 1970; Itin & Hehl, 2013; Itin & Hehl, 2015) has so far led to the following. The tensor C=S+A is decomposed (see, e.g., Backus, 1970) into a fully symmetric part S (Equation 3 above), and its asymmetric (not antisymmetric) complement A, as shown in FIG. 1a:

$${C}_{ijkl} = {S}_{ijkl} + {A}_{ijkl} , \qquad ( 5 )$$

$${S}_{ijkl} = {C}_{( ijkl )} = 1 / 3 ( {C}_{ijkl} + {C}_{iljk} + {C}_{ikjl} ) ,$$

$${A}_{ijkl} = {C}_{ijkl} - {C}_{( ijkl )} = 1 / 3 ( 2 {C}_{ijkl} - {C}_{iljk} - {C}_{ikjl} ) .$$

Tensor S (and kurtosis tensor, Equation (3)) further splits into irreducible representations of SO(3) with [symbol] =0, 2, 4:

$$S = {S}^{( 0 )} + {S}^{( 2 )} + {S}^{( 4 )} , \qquad ( 6 )$$

where S⁽⁰⁾ is fully isotropic ( [symbol] =0) and defined by one dof (the full trace Sᵢᵢⱼⱼ of S), whereas S⁽²⁾ and S⁽⁴⁾ are symmetric trace-free (STF) tensors that are parametrized by 5 and 9 dof, respectively, with the total dof count 15=1+(2·2+1)+(2·4+1) (see Materials and Methods).

Importantly, tensor A (6 dof=21−15) is equivalent to a symmetric 3×3 tensor (see, e.g., Itin & Hehl, 2013):

$${A}_{pq} = {ϵ}_{ikp} {ϵ}_{jlq} {A}_{ijkl} = {δ}_{pq} ( {A}_{iikk} - {A}_{ikik} ) + 2 ( {A}_{pkqk} - {A}_{pqkk} ) , \qquad ( 7 )$$

$${A}_{ijkl} = 1 / 6 ( {ϵ}_{ikp} {ϵ}_{jlq} + {ϵ}_{ilp} {ϵ}_{jkq} ) {A}_{pq} ,$$

where δᵢⱼ is Kronecker's delta, and ϵᵢⱼₖ is the fully antisymmetric Levi-Civita tensor. Therefore, A splits into irreducible representations with [symbol] =0 and 2, FIG. 1a:

$$A = {A}^{( 0 )} + {A}^{( 2 )} . \qquad ( 8 )$$

Hence, non-LTE B-tensor shapes (see, e.g., Westin et al., 2016) probe the A tensor. Specifically, STE is sensitive to the isotropic ( [symbol] =0) part A⁽⁰⁾, while more general B-tensor shapes, such as PTE, also probe the [symbol] =2 part A⁽²⁾, as shown in FIG. 1b and Eq. (9) indicated herein below.

### Exemplary Geometric Meaning

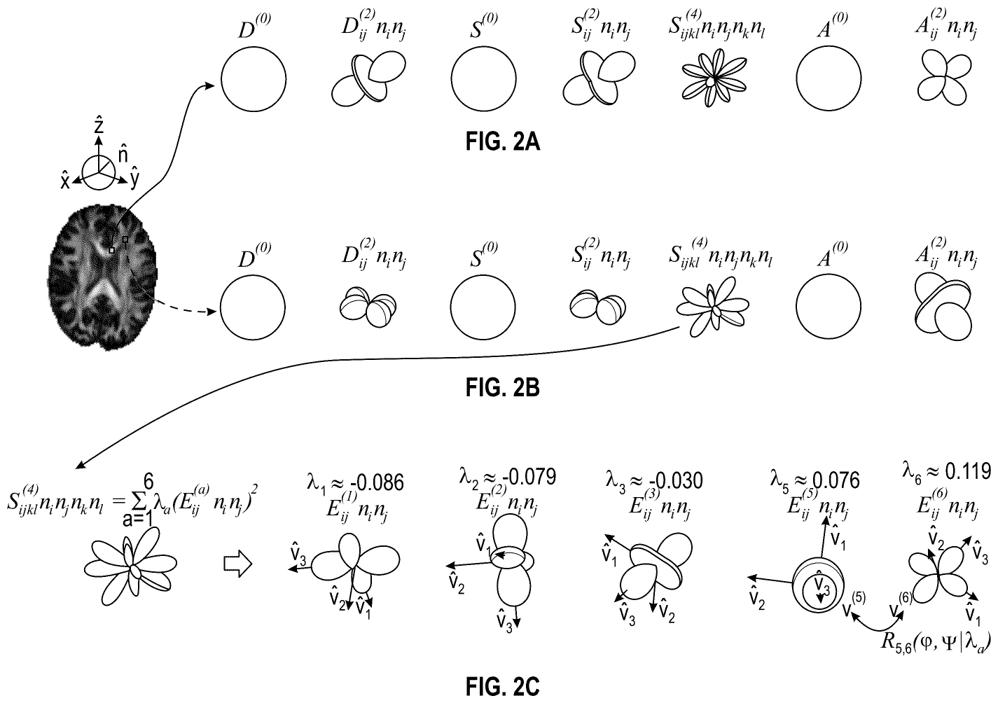

The correspondence between symmetric trace-free (STF) tensors and spherical harmonics (SH) (see, e.g., Eq. (19) herein below) indicates that, if the tensor glyph S(\hat{n})=Sᵢⱼₖₗ nᵢnⱼnₖnₗ is drawn, where n is a unit vector, then the S⁽⁰⁾ part gives the directional average of S(\hat{n}) (a ball). The S⁽²⁾ part is responsible for a “single-fiber” glyph parametrized by five Y^{2m}(\hat{n}) (turning the ball into an ellipsoid, much like D(\hat{n})=Dᵢⱼnᵢnⱼ in diffusion tensor imaging, or DTI). The S⁽⁴⁾ part, parametrized by nine Y^{4m}(\hat{n}), depicts multiple lobes coming from fiber crossings, as shown in FIG. 2.

Among the 5 dof of S⁽²⁾, 3 absolute angles can define the orientation of the glyph. The remaining 2 dof parametrize the three eigenvalues that sum up to zero, as tr S⁽²⁾=0. Thereby, the 3 eigenvalues of S⁽⁰⁾+S⁽²⁾ (one coming from S⁽⁰⁾ and two from S⁽²⁾) are the invariants that determine the semi-axes of an ellipsoid.

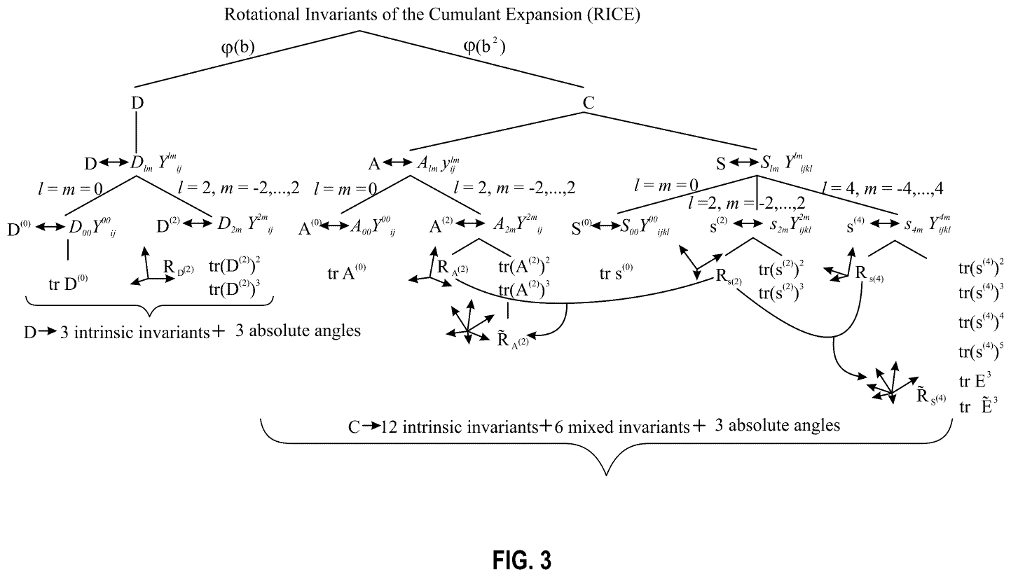

For S⁽⁴⁾, the picture is more complex. The 3 absolute angles again determine the orientation of the object Sᵢⱼₖₗ⁽⁴⁾nᵢnⱼnₖnₗ, while the remaining 6 dof are the invariants determining its shape (see FIG. 3).

Similarly to S⁽⁰⁾+S⁽²⁾ or DTI examples above, the A tensor can describe a “single-fiber” glyph, with its 1+2 invariants parametrizing the semi-axes of the corresponding ellipsoid, and the remaining 3 dof determining its orientation in space. In particular, for axially symmetric B-tensors, Equation (4), the contribution of the A tensor to the second term in the cumulant expansion (1),

$$\frac{1}{2} {B}_{ij} {B}_{kl} {A}_{ijkl} = \frac{1}{27} {b}^{2} ( 1 - β ) ( 1 + 2 β ) {A}_{pp} - \frac{1}{9} {b}^{2} β ( 1 - β ) {A}_{pq} {g}_{p} {g}_{q} , \qquad ( 9 )$$

can be expressed as a scalar ∝Aₚₚ=(3/2) Aᵢᵢₖₖ (full trace), plus an ellipsoid parametrized by the tensor (7a). As expected, Eq. (9) vanishes for LTE (β=1). For STE, only the first (scalar) term survives, whereas PTE is the cleanest encoding to probe the A tensor ellipsoid: the first term vanishes, and the second term yields (b²/12) A_{pq}gₚg_{q}.

### Exemplary Invariants: Intrinsic and Mixed

The representation theory (see, e.g., Backus, 1970; Itin & Hehl, 2013; and Itin & Hehl, 2015), while elegant, has so far not been tied to constructing the invariants. Here we reconstruct all invariants of C by combining the characteristic polynomial approach (see, e.g., Betten, 1987; and Basser & Pajevic, 2007) with the representation theory (see, e.g., Backus, 1970; and Itin & Hehl, 2013; Itin & Hehl, 2015). Namely, according to exemplary embodiments of the present disclosure, it is possible to construct the invariants for each representation of

$$C = {S}^{( 0 )} + {S}^{( 2 )} + {S}^{( 4 )} + {A}^{( 0 )} + {A}^{( 2 )} \qquad ( 10 )$$

separately (see FIG. 3). This can also apply to the overall diffusion tensor

$$D = {D}^{( 0 )} + {D}^{( 2 )} . \qquad ( 11 )$$

It is then possible to complement these intrinsic invariants with the mixed ones defining the relative orientations between irreducible components of C with [symbol] >0.

For example, the total number of invariants for any tensor equals to its number of dof minus 3 absolute rotation angles (see, e.g., Ghosh et al., 2012), yielding 3 for D (DTI), 12 for S or W tensors (DKI), and 18 for C. Applying this argument to each irreducible representation, the number of intrinsic invariants is 1 for [symbol] =0, and (2 [symbol] +1)−3=2( [symbol] −1)=2, 6, . . . for [symbol] =2, 4, . . . . The relative angles between irreducible components likely do not change upon rotation of the basis (the tensor transforms as a whole). Therefore, the 21 dof of C are split between (1+2+6)+(1+2)=12 intrinsic invariants according to decomposition (10); 3+3=6 mixed invariants (relative angles between components A⁽²⁾, S⁽²⁾, and S⁽⁴⁾); and 3 absolute rotation angles, as shown in FIG. 3 and described herein.

### Relation to Tissue: Exemplary Addition of Angular Momenta

Eq. (2) can be reviewed through the lens of representation theory. In particular, the right-hand side of (2) involves a direct product of two compartment tensors, which can symbolically denote D⊗D (dropping compartment index α for brevity). Each D=D⁽⁰⁾+D⁽²⁾ can be split into [symbol] =0 and 2 representations (e.g., by going into the STF tensor basis, as shown in FIG. 1d). The resulting direct products

$$D ⊗ D = {D}^{( 0 )} ⊗ {D}^{( 0 )} + {D}^{( 0 )} ⊗ {D}^{( 2 )} + {D}^{( 2 )} ⊗ {D}^{( 0 )} + {D}^{( 2 )} ⊗ {D}^{( 2 )} \qquad ( 12 )$$

correspond to the addition of two angular momenta with [symbol] =0 or [symbol] =2.

As it is known from quantum mechanics (see, e.g., Tinkham, 2003), the addition of angular momenta [symbol] + [symbol] yields all possible momenta between | [symbol] ₁− [symbol] ₂| and [symbol] ₁+ [symbol] ₂. Mathematically speaking (see, e.g., Hall, 2015), the tensor product of representations [symbol] ₁ and [symbol] ₂ is reducible, and splits into a direct sum of irreducible representations with [symbol] =| [symbol] ₁− [symbol] ₂|, . . . , [symbol] ₁+ [symbol] ₂.

The successive terms in Eq. (12) yield: 0⊗0=0; 0 ⊗2=2 ⊗0=2; 2⊗2=0⊕2⊕4. In the latter case, representations with [symbol] =1 and 3 forbidden by parity were not included. Hence, the right-hand side of Eq. (2) is a direct sum of representations 0⊕2⊕0⊕2⊕4. This is precisely what we get from Eq. (10) representing the left-hand side of Eq. (2).

Thus, importantly, according to the exemplary embodiments of the present disclosure, the representations of SO(3) group can dictate the relations between distinct components of the C tensor and tissue properties. Furthermore, the coefficients with which the STF component covariances [symbol] (see FIG. 1 e) enter the STF components of the S and A tensors, are the Clebsch-Gordan coefficients (see, e.g., Tinkham, 2003) which obey the selection rules from the addition of angular momenta as discussed herein.

### Exemplary MD, FA, MK and μFA from RICE

For example, not all 3+18 RICE invariants are equally important. According to an exemplary embodiments of the present disclosure, it is possible to identify, e.g., 7 of the invariants (e.g., 2 from D and 5 from C) which are related to previously studied contrasts. Such invariants all correspond to L₂-norms of the representations of C and D, Eqs. (10) and (11). These RICE invariants can be called the main RICE invariants, and a special notation for them can be introduced, as they play a unique and/or important role in synthesizing dMRI contrasts:

$${D}_{0} = \frac{1}{3} tr D , {D}_{2}^{2} = \frac{2}{3} tr {( {D}^{( 2 )} )}^{2} \qquad ( 13 )$$

$${S}_{0} = \frac{1}{5} tr S , {S}_{2}^{2} = \frac{4}{7} tr {( {S}^{( 2 )} )}^{2} , {S}_{4}^{2} = \frac{8}{35} tr {( {S}^{( 4 )} )}^{2} ,$$

$${A}_{0} = \frac{1}{3} tr A , {A}_{2}^{2} = \frac{2}{3} tr {( {A}^{( 2 )} )}^{2}$$

Interestingly, it is possible to express conventional scalar contrasts—mean diffusivity (MD), fractional anisotropy (FA), mean kurtosis (MK), and microscopic fractional anisotropy (μFA)—in terms of D₀, D₂, S₀, and A₀:

$$MD = {D}_{0} , FA = \sqrt{\frac{3 {D}_{2}^{2}}{4 {D}_{0}^{2} + 2 {D}_{2}^{2}}} , \qquad ( 14 )$$

$$MK = \frac{3 {S}_{0}}{{D}_{0}^{2}} , μ FA = \sqrt{\frac{60 {S}_{0} - 30 {A}_{0} + 27 {D}_{2}^{2}}{40 {S}_{0} - 20 {A}_{0} + 18 {D}_{2}^{2} + 36 {D}_{0}^{2}}} .$$

Further, axial and radial kurtosis for an axially-symmetric fiber tract can be expressed via D₀, D₂, S₀, S₂ and S₄, without the need to rotate to the basis aligned with the tract. Further, it is possible to also identify a previously unexplored contrast coming from a combination of S⁽²⁾ and A⁽²⁾ elements, which can be used to define the size-shape correlation index of compartmental tensors.

### Exemplary Minimal Protocols: iRICE

Table 1 shows the exemplary minimal instant/icosahedral RICE (iRICE) protocols to obtain MD, FA, MK, based on measurements with only 12 B tensors; and MD, FA, MK, μFA based on measurements with only 15 B tensors, in accordance with certain exemplary embodiments of the present disclosure. These numbers can notably be fewer than 21=6+15 or 27=6+21 necessary to determine all tensor components. These acquisition schemes can involve fewer measurements than was previously anticipated.

TABLE 1
Comparison among existing and proposed fast protocols.
Exemplary minimal protocols contain the minimum unique
number of directions and distinct b-values (exemplary
b-values can be altered). For STE, more than 1 direction
implies rotation of the waveform for accuracy. The
protocols can include a non-diffusion weighted image (b = 0).
Fast protocols comparison, b-values are in ms/μm²

| | | |
|---|---|---|
| Output maps | MD + MK | MD + FA + MK |
| Theoretical minimum | 6 × LTE_{b=2} | 6 × LTE_{b=1}; 6 × LTE_{b=2} |
| iRICE (MD + FA + MK) | 1 × STE_{b=1} | 6 × LTE_{b=1}; 6 × LTE_{b=2} |
| Hansen et al. (2013) | 3 × LTE_{b=1}; 9 × LTE_{b=2} | |
| Output maps | MD + MK + μFA | MD + FA + MK + μFA |
| Theoretical minimum | 6 × LTE_{b=2} | 6 × LTE_{b=1}; 6 × LTE_{b=2} |
| | | 1 × STE_{b=1.5} |
| iRICE (MD + FA + MK + μFA) | 1 × STE_{b=1}; 1 × STE_{b=1.5} | 6 × LTE_{b=1}; 6 × LTE_{b=2} |
| | | 3 × STE_{b=1.5} |
| Nilsson et al. (2020) | 3 × LTE_{b=0.1}; 3 × LTE_{b=0.7}; 6 × LTE_{b=1.4}; 6 × LTE_{b=2} | |
| | 6 × STE_{b=0.1}; 6 × STE_{b=0.7}; 10 × STE_{b=1.4}; 16 × STE_{b=2} | |

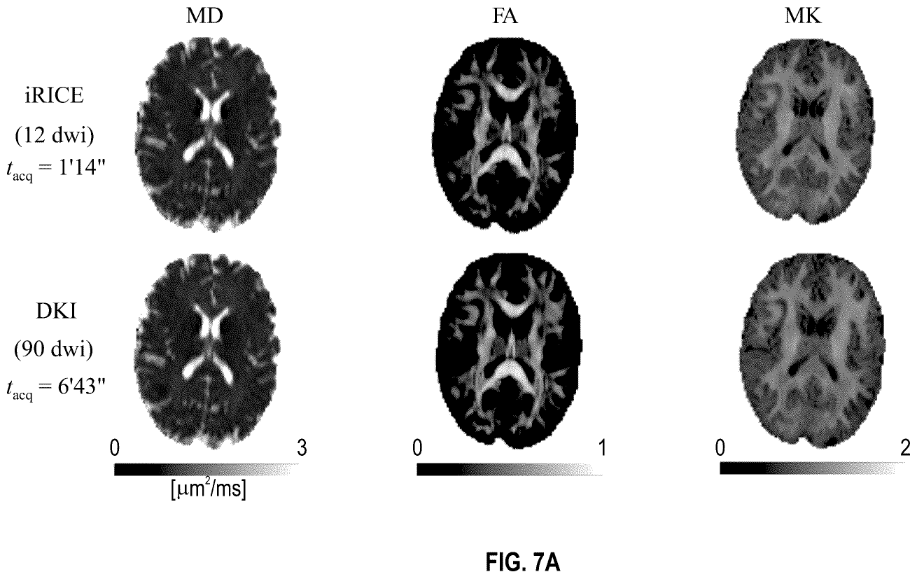

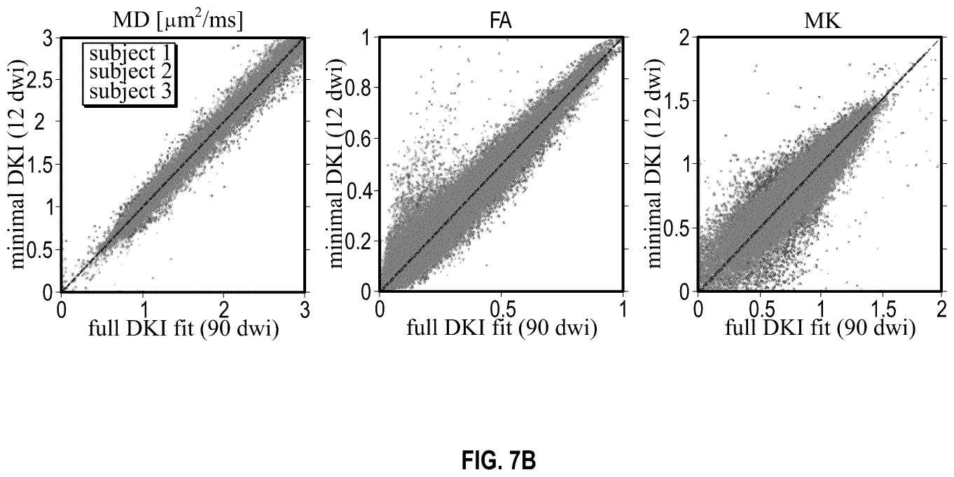

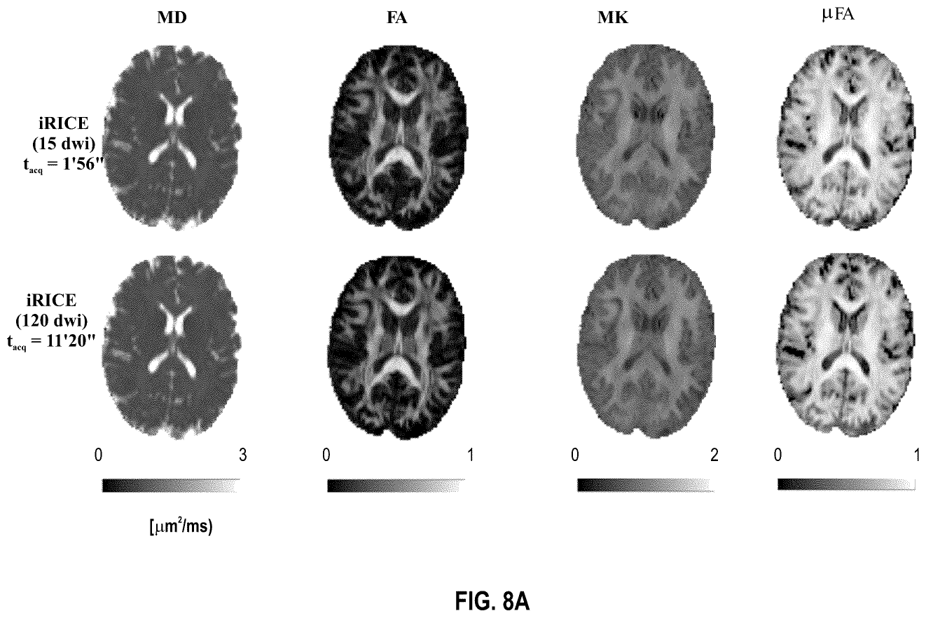

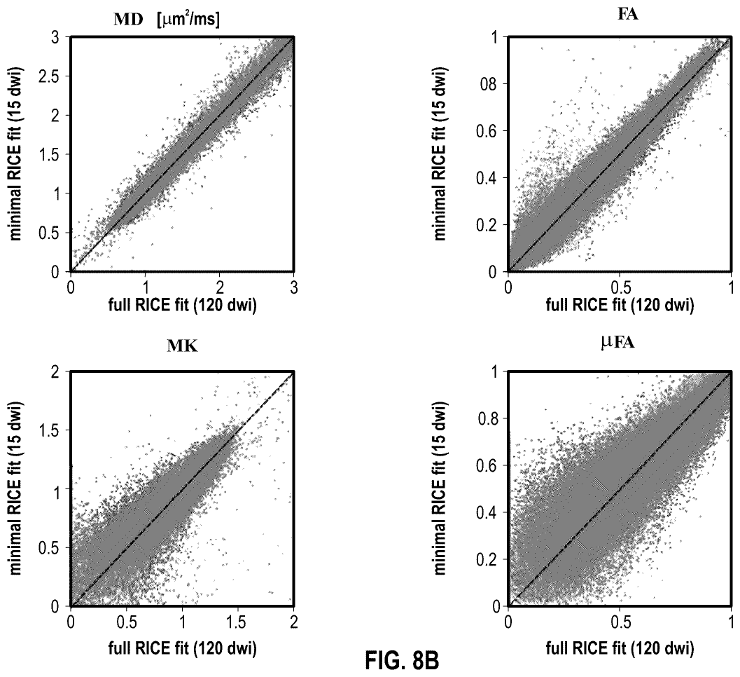

As described herein, it is possible to use spherical designs (special directions on a sphere) which can, e.g., guarantee that relevant tensor components are estimated, whereas the inessential ones (that do not contribute to the above main invariants) are canceled. FIGS. 7 and 8 show and compare exemplary parametric maps iRICE and fully sampled protocols. It is possible to prove that the minimal protocols can be based on icosahedral directions for which the tensor elements not involved in MD, FA, MK, μFA cancel, as discussed for the exemplary embodiments herein.

### Exemplary In Vivo RICE Maps and Exemplary Comparison with iRICE

A systematic derivation of all rotational invariants of the cumulant expansion (RICE) from an irreducible decomposition of the diffusion and covariance tensors can be provided, in accordance with the exemplary embodiments of the present disclosure, as shown in FIGS. 2a-2c and 3. With this exemplary decomposition, it is possible to rewrite the covariance tensor in STF basis, facilitating a direct link between its degrees of freedom and the type of information present: size variance, shape variance, size-shape covariance, as shown in the diagram of FIG. 1e.

For example, FIG. 2b shows the irreducible decompositions of C and D tensors, Eqs. (10) and (11), for a white matter voxel of the longitudinal superior fasciculus crossing fibers—Glyphs are rescaled to a similar size and color-coded by the sign (red=positive), while radius represents the absolute value. Further, FIG. 2c shows a representation of the eigentensor decomposition, Eq. (29), for the crossing fiber voxel shown in FIG. 2b (here λ₄=0, Eᵢⱼ⁽⁴⁾=δᵢⱼ/\sqrt{3}). Two dof control the relative orientations among any pair of Eᵢⱼ^{(a)}, and their relative orientations are coupled since 5 degrees of freedom define the full orientation of the set of eigentensors. FIG. 3 shows the full irreducible decomposition of C and D tensors in STF basis, Eqs. (10) and (11). Each irreducible component has its intrinsic invariants (1 for [symbol] =0; 2 for [symbol] =2; and 6 for [symbol] =4). Together with these 1+2+1+2+6=12 intrinsic invariants, C has 9 basis-dependent absolute angles defining the orientations of its S⁽²⁾, S⁽⁴⁾, and A⁽²⁾ components via the rotation matrices [symbol] _{S}_{⁽²⁾}, [symbol] _{S}_{⁽⁴⁾}, and [symbol] _{A}_{⁽²⁾}, such that total dof count of C is 21=12+9. Out of 9 angles, 6 mixed invariants of C correspond to 6 relative angles between A⁽²⁾, S⁽²⁾ and S⁽⁴⁾. Here, as an example, we take [symbol] _{S}_{⁽²⁾} as a reference basis, and compute the relative orientations [symbol] _{S}_{⁽⁴⁾} and [symbol] _{A}_{⁽²⁾}.

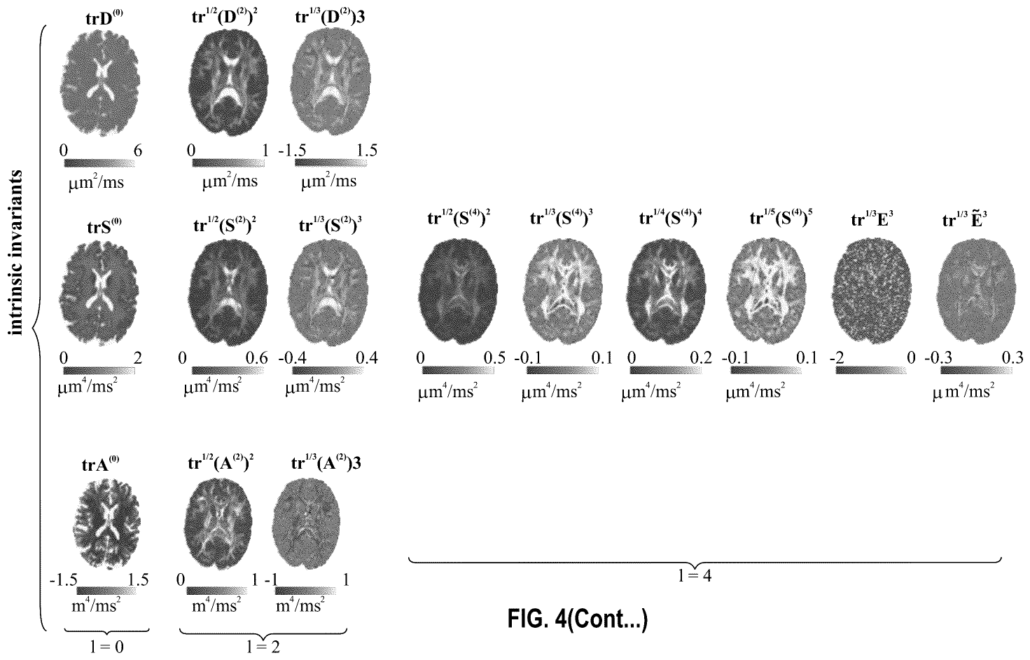

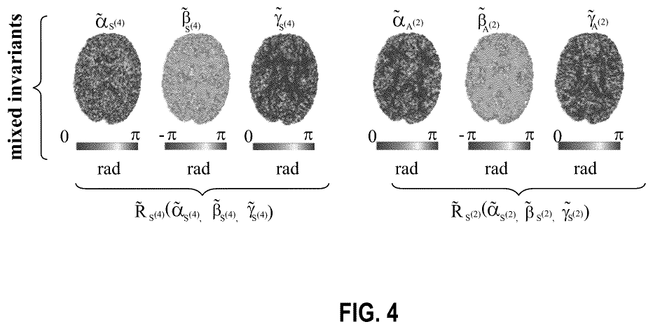

With RICE, we uncover multiple diffusion contrasts that have been overlooked so far. We separate all rotational invariant information contained in D and C tensors, as shown in the exemplary maps of FIG. 4. The main exemplary RICE maps, ∝tr^{1/2}( [symbol] )², e.g., are the only ones whose combinations have been studied in the literature, since these together are equivalent to standard DTI+DKI+μFA contrasts. Furthermore, from these it is also possible to define a novel size-shape correlation (SSC) invariant, in accordance with the exemplary embodiments of the present disclosure. Indeed, FIG. 4 shows all RICE maps for a normal brain (33 y.o. male). Different combinations of these invariants contribute to conventional diffusion contrasts. Rotation matrices [symbol] describing mixed invariants are parametrized by Euler angles of the bases of S⁽⁴⁾ and A⁽²⁾ sectors relative to the eigenbasis of S⁽²⁾. These angles correspond to intrinsic (active) rotations of the S⁽²⁾ frame along z by \tilde{α}, then along new x′ by \tilde{β}, and along new z″ by \tilde{γ} (equivalent to the product of extrinsic active rotations [symbol] (\tilde{α},\tilde{β},\tilde{γ})=X(\tilde{α})Y(\tilde{β})Z(\tilde{γ}) in the fixed S⁽²⁾ frame to obtain the S⁽⁴⁾ and A⁽²⁾ frames). Underlying tissue microstructure introduces correlations between invariants. For example, the near-zero relative angles \tilde{β} in the white matter tracts exemplify the alignment of the eigen frames from different representations with the local tract orientation.

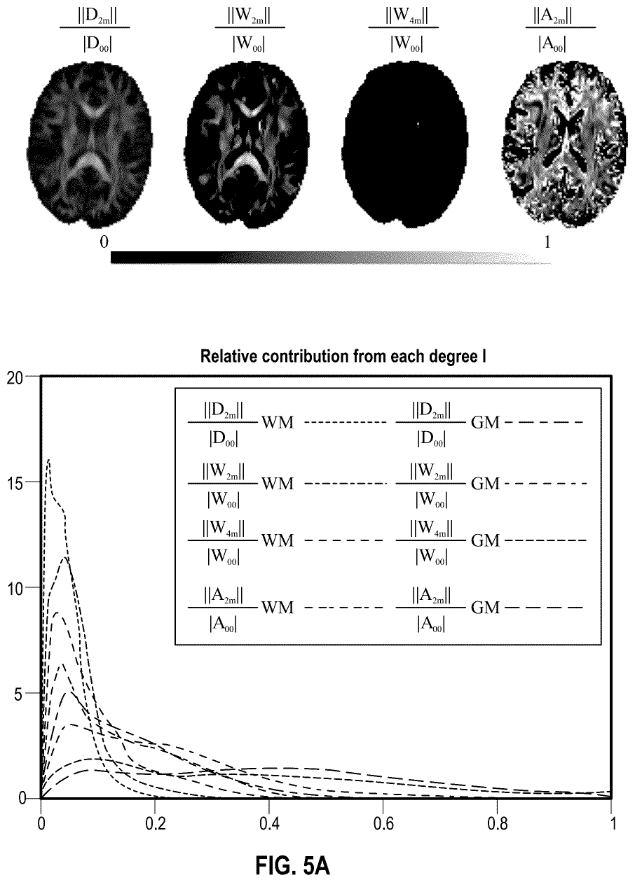

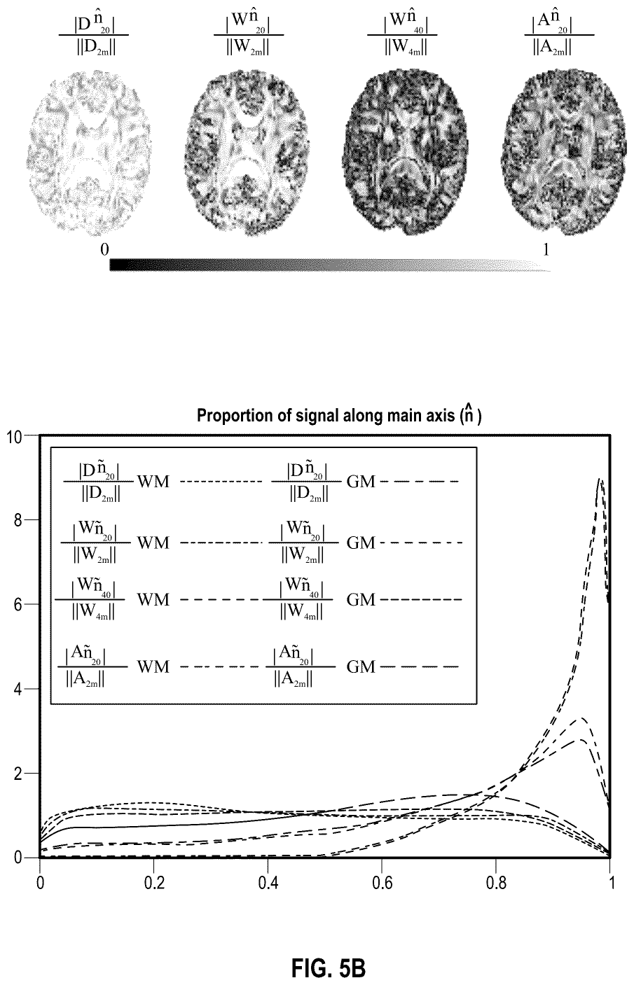

FIG. 5a shows exemplary maps of the ‘energy’ ratios of [symbol] =2 and [symbol] =4 invariants relative to [symbol] =0, together with histograms containing gray and white matter voxels. It is evident that the contribution of high-order information in D, W, and A decreases with the degree e for both tissues. This is more pronounced in gray matter and less so in crossing fibers or highly aligned WM regions, e.g., the corpus callosum. In the exemplary maps of FIG. 5b, all voxels' principal fiber axis \hat{n} is rotated towards \hat{z}, and the relative fraction of ‘energy’ is computed or otherwise determined along such axis. This is an exemplary measure of axial symmetry for each degree [symbol] , since axially symmetric tensors satisfy | [symbol] |/ [symbol] ≡1 for all [symbol] . The above shows that D⁽²⁾ and W⁽²⁾ have a high axial symmetry, while the opposite holds for W⁽⁴⁾ and A⁽²⁾. For axially symmetric tensors these ratios equal 1. In the kurtosis tensor, the 9 elements associated with [symbol] =4 have a 5-10 times smaller intensity than the trace. This empirical observation about the smallness of S⁽⁴⁾ component of the fully-symmetric part S of covariance tensor can be used for making the estimation of components of D, S and C tensors more robust, such as, but not limited to, imposing the constraint on the component S⁽⁴⁾ to be small, as further discussed herein.

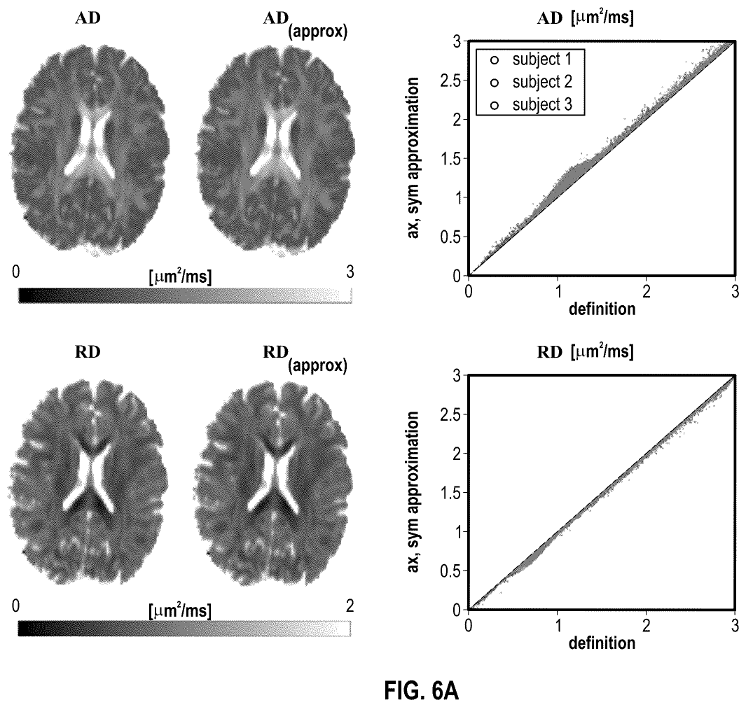

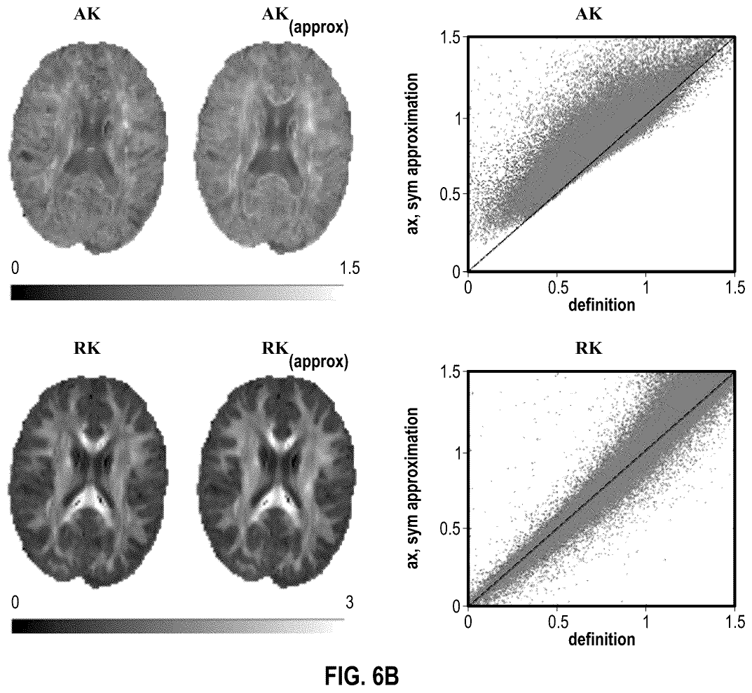

The bottom rows of FIGS. 6a and 6b shows typical projections of the diffusion and kurtosis tensors onto the principal fiber basis, Eq. (42). These projections have a clear physical meaning when there is a predominant fiber in a voxel, where they are dubbed as axial or radial. The top rows of FIGS. 6a and 6b show an approximation of such maps, Eq. (43), that do not involve projecting to the principal fiber basis. This expression is exact if the tensors are axially symmetric, and results in a good agreement in the whole brain even when this condition does not hold. This happens because, although W₄ₘ are not axially symmetric, they are much smaller than W₀₀ and W₂ₘ.

Minimal and fully sampled acquisitions were thoroughly compared. Exemplary maps for MD+FA+MK are shown in FIG. 7a. All pairs of the exemplary maps look qualitatively similar and scatter plots do not show biases. Regions where W₄ₘ elements are larger, e.g., WM fiber crossings, show good correspondence in MK maps. FIG. 7b shows scatter plots that include whole brain voxels from 3 normal volunteers comparing MD, FA, and MK from iRICE vs fully sampled RICE maps. Similarly, minimal and fully sampled maps for MD+FA+MK+μFA are shown in FIG. 8a. In both cases, minimal maps showed a very similar contrast to the fully sampled ones despite having 8-fold fewer measurements and not estimating the full tensors involved in each representation. FIG. 8b shows scatter plots that include whole brain voxels from 3 normal volunteers comparing MD, FA, MK, and μFA from iRICE vs fully sampled RICE maps.

### FURTHER EXEMPLARY DISCUSSION

### Exemplary RICE

The main exemplary RICE maps, ∝tr^{1/2}( [symbol] )², are related to conventional contrasts, Eqs. (36)-(39), without assumptions on the tissue diffusion tensor distribution [symbol] (D). An advantage of RICE maps over other signal representations can be that they belong to distinct irreducible representations of rotations, and thus, represent “orthogonal” (complementary) contrasts up to [symbol] (b²). RICE parameters thereby can be used to represent a hardware- and measurement-independent “fingerprint” of the tissue (or, more generally, of a physical structure under consideration) in a mutually complementary way, without any further assumptions. RICE maps (i.e., RICE parameters determined for a number of voxels in an organ or tissue) can be used as inputs to machine-learning or artificial-intelligence classifiers, such as, but not limited to, neural networks, trained for an automatic pathology detection and staging. The particular advantage of such RICE parameter maps as inputs for machine-learning classifiers is in their independence of the hardware and measurement protocol; this enables combining training data from multiple sites, scanner manufacturers, field strengths, and other differing tissue-independent characteristics, that could otherwise limit the effectiveness of training of said classifiers.

There is certain previously-unexplored information, complementary to μFA, in the non-symmetric part of C. It is related to the size-shape correlation of compartmental diffusion tensors. The complementary information needed for such contrast is present in the anisotropic part A⁽²⁾ of A. This is inaccessible by LTE+STE acquisitions, although can be measured combining LTE and PTE, as shown in the diagram of FIG. 1b.

For example, not all RICE invariants may be equally important. Some can reflect more prominent signal features and thus show higher SNR maps. Furthermore, although exemplary RICE invariants can be mathematically independent, it was determined, according to the exemplary embodiments of the present disclosure, that some correlations exist between them. This highlights that brain tissue [symbol] (b²) signals may not have independent 18 rotationally invariant dof. For example, in white matter, the mixed invariants provided in the exemplary maps of FIG. 4 show relative Euler angles \tilde{β}_{S}_{⁽⁴⁾} and \tilde{β}_{A}_{⁽²⁾} near zero, exemplifying the alignment between the frames of different irreducible representations along the principal fiber direction. This empirical observation can be further used to impose an alignment constraint between the eigenbases of different parts of the S, A and D tensors, to make parameter estimation from dMR measurements more robust, precise and/or accurate. Such exemplary constraint can be imposed, for example, as a constraint for scalar products between corresponding eigenvectors of S2, S4, A and D tensors, which can lead to an optimization problem for finding the remaining elements of D and C tensors.

The STF basis allowed the analysis of the different degrees of anisotropy in D, W (or S), and A. It was observed in both gray and white matter that the relative contributions from degrees [symbol] =0,2,4 are not equally important. As [symbol] becomes higher, tensor elements become smaller (as shown in FIGS. 5a and 5b). It was also observed that axial asymmetry arises mostly from [symbol] =4 elements in kurtosis while it is practically absent in the diffusion tensor or in [symbol] =2 kurtosis elements. This facilitates a fast approximate computation of axial and radial diffusion/kurtosis without the need to project D and W onto the main eigenvector of D.

A comparison between fiber-basis projection maps (axial and radial diffusion/kurtosis) and axial symmetry approximations shows high agreement (see, e.g., FIGS. 6a and 6b). Such empirical observations can support approximations of axisymmetric W (Hansen et al., 2016), such that only m=0 element of S⁽⁴⁾ (equivalently, of W⁽⁴⁾) is nonzero. This may also enable fast quantification of axial and radial kurtosis.

Furthermore, the empirical observation of smallness of S₄ (equivalently, of W₄) as compared to [symbol] =0 and 2 components, can lead to improvement of precision, accuracy and/or robustness of estimating the tensor components, by constraining all elements of S₄ₘ (equivalently, of W₄ₘ) to be small; and/or setting those elements to zero during estimation. For example, such smallness constraint can be imposed by using a corresponding training set with small or zero S₄ₘ, when learning the mapping between the measurements and the tensor components of D and C tensors.

Throughout the disclosure of the exemplary embodiments, MK can be referred to as, e.g., the trace of the kurtosis tensor, as defined in Eq. (37) (see, e.g., Hansen et al., 2013; Jespersen, 2018). Such definition has two main advantages over the definition using the average of the directional apparent kurtosis: First, it is a tensor derived quantity, the rotational invariant [symbol] . Second, estimating it from noisy measurements is a better-posed inverse problem presenting fewer outliers (“black voxels”).

### Exemplary iRICE

According to certain exemplary embodiments of the present disclosure, two acquisition protocols minimizing the number of directions for MD, FA, MK, and μFA can be provided. For example, using STF decompositions of the diffusion, kurtosis, and covariance tensors, we separate the contribution of each tensor element to the parametric maps of interest. By canceling unnecessary contributions, e.g., it is possible to reduce the number of free parameters, allowing the estimation of different contrasts combinations in a faster scan. The exemplary minimal acquisitions, in accordance with the certain exemplary embodiments of the present disclosure, can include, e.g., 6×LTE_{b=1}+6×LTE_{b}=₂=12 DWIs for MD+FA+MK in a 1-minute scan, and 6×LTE_{b=1}+3×STE_{b}=1.5+6×LTE_{b}=₂=15 DWIs for MD+FA+MK+μFA in a 2-minute scan, see Table 2. For example, as discussed herein, DWI can refer to diffusion-weighted MR image, or measurement. The feasibility of exemplary minimal protocols is provided on a normal volunteer. The smallest spherical designs facilitating a joint fit are used but the robustness and precision of both minimal protocols can be enhanced by using spherical designs that have more directions, e.g., full icosahedrons for each shell.

Table 1 shows a comparison between conventional fast protocols and the minimal fast protocols, in accordance with the exemplary embodiments of the present disclosure. One publication (see, e.g., Hansen et al., 2013) suggested a fast DKI approach of 3×LTE_{b=1}+9×LTE_{b=2}=12 DWI, relying on a two-step fitting rather than a joint one. This is suboptimal due to nonzero contributions of W elements at b=1 ms/μm², which bias MD and MK estimation. Nonetheless, the same or similar estimation approach can be applied to the framework, in accordance with the exemplary embodiments of the present disclosure, with only 1×STE_{b}1+6×LTE_{b}=₂=7 DWI or 3×LTE_{b=1}+6×LTE_{b}=₂=9 DWI. Similarly, another publication (see, e.g., Nilsson et al., 2020) proposed a 3-minute protocol for extracting MD, MK, and μFA. In such conventional approach, thicker slices and more measurements are used, but the same direction sets to compute spherical means. These were used to fit a moments-based approximation involving diffusion isotropic and anisotropic variances from which MK and μFA can be computed. However, this may not facilitate a simultaneous estimation of FA since such expression only holds for spherical means. Recently, a further publication (see, e.g., Kerkela et al., 2021) used Monte Carlo simulations to show that computing μFA from the estimation of the cumulants D and C has a minimum finite-b bias. Due to this, protocols, in accordance with the exemplary embodiments of the present disclosure, can facilitate a joint fit of the cumulants from directional signals rather than from spherical means.

### Exemplary Materials and Methods

### Exemplary Multiple Gaussian Compartments

In general, the dMRI signal depends on all sequence timings and the corresponding Larmor frequency gradient values g(t); mathematically, the signal is a functional of g(t), or, equivalently, of the encoding function q(t)=∫₀^{t}g(t′)dt′ (the anti-derivative of g(t)): S=S[q(t)]. Even for a conventional pulsed-gradient diffusion sequence (missing citation), one obtains a multi-dimensional phase diagram in the space of sequence parameters (see, e.g., Kiselev, 2021; and Novikov, 2021).

However, when each compartment is fully coarse-grained, the measurement is completely determined by the B-tensor with elements

$${B}_{ij} = {\int }_{0}^{TE} {q}_{i} ( t ) {q}_{j} ( t ) dt , b = tr B , \qquad ( 15 )$$

calculated based on q(t). The distribution [symbol] (D) of compartment tensors in a given voxel gives rise to the overall signal

$$S = {\int }_{}^{} dD 𝒫 ( D ) {e}^{- tr BD} = {\sum }_{α} {f}_{α} {e}^{- tr {BD}^{α}} , \qquad ( 16 )$$

where D^{α} are compartment diffusion tensors (see, e.g., Basser & Pajevic, 2003; Jian et al., 2007; and Glenn et al., 2015). Normalization ∫dD [symbol] (D)=1 implies that the fractions add up to unity, Σ_{α}ƒ_{α}=1.

Equation (16) can be the most general form of a signal from multiple Gaussian compartments. It is valid when the transient processes have played out, such that tensors D^{α} have all become time-independent, and thereby higher-order cumulants in each compartment are negligible (see, e.g., Novikov et al., 2019). In this case, the signal (16) is a function of the B-tensor: S[q(t)]→S(B), while tissue is fully represented by the distribution [symbol] (D). This long-t picture of multiple Gaussian compartments (anisotropic and non-exchanging) underpins a large number of dMRI modeling approaches, in particular, the Standard Model (SM) of diffusion (see, e.g., Novikov et al., 2019) and its variants (see, e.g., Jespersen et al., 2007; Jespersen et al., 2010; Fieremans et al., 2011; Zhang et al., 2012; Sotiropoulos et al., 2012; and Jensen et al., 2016). Furthermore, this picture contains the SM extension onto different fiber populations in a voxel, lifting the key SM assumption of a single-fascicle “kernel” (response).

Given the forward model (16), an inverse problem is to restore ?(D) from measurements with different W. This problem is a matrix version of the inverse Laplace transform and is therefore ill-conditioned. Since in clinical settings, typical encodings are moderate (tr BD_{α}˜1), the inverse problem can be formulated term-by-term for the cumulant expansion (1) of the signal (16).

### Exemplary Parameter Count in Cumulant Series

The higher-order signal terms in Eq. (1) couple to successive cumulants of ?(D). The inverse problem maps onto finding the cumulants [symbol] Dᵢ_{₁}ⱼ_{₁}^{α} . . . Dᵢ_{₁}ⱼ_{₁}^{α} [symbol] (tensors of even order 2n) from a set of measurements. This becomes obvious by noting the analogy B→iλ with the standard cumulant series (see, e.g., van Kampen, 1981) ln

$$p ( λ ) = {\sum }_{n = 1}^{∞} \frac{{( - i λ )}^{n}}{n !} 〈 〈 {x}^{n} 〉 〉$$

for the characteristic function p(λ)=∫dx e^{-iλx}p(x) of a probability distribution p(x), such that the n-th term in Eq. (1) is

$$\frac{{( - )}^{n}}{n !} {B}_{{i}_{1} {j}_{1}}^{} … {B}_{{i}_{n} {j}_{n}} 〈 〈 {D}_{{i}_{1} {j}_{1}}^{α} … {D}_{{i}_{n} {j}_{n}}^{α} 〉 〉 .$$

Hence, the B-tensor lowers the order by half: The 2n-th diffusion-displacement cumulant (the 2n-th order term from expanding ln S[q(t)] in q(t)) maps onto the n-th cumulant [symbol] Dᵢ_{₁}ⱼ_{₁}^{α} . . . Dᵢ_{ₙ}ⱼ_{ₙ}^{α} [symbol] of [symbol] (D) corresponding to the n-th order of expansion of InS(B) in B. The number of dof for this cumulant equals to that for a fully symmetric order-n tensor of dimension d=6, which is a number of assignments of n indistinguishable objects into d distinguishable bins: (n+d−1)!/n!(d−1)!=(n+5)!/n!5!=6, 21, 56, 126, . . . for n=1, 2, 3, 4, . . . .

### Exemplary Representations of SO(3)

The exemplary method of analysis, in accordance with the exemplary embodiments of the present disclosure, can utilize the representation theory of SO(3), the group of rotations in 3 dimensions (see, e.g., Tinkham, 2003; and Hall, 2015). A d-dimensional representation of a group is a mapping of each element (rotation) onto a d×d matrix that acts on a d-dimensional vector space. Representation theory provides a way to split a complex object (such as tensor D or C) into a set of independent simpler ones with certain symmetries, on which a group acts. In particular, the elements of an irreducible representation transform among themselves, and hence can be reviewed separately.

All irreducible representations of SO(3) are labeled by integers [symbol] =0, 1, 2, . . . , and have dimension 2 [symbol] +1. Each representation is equivalent to a set of 2 [symbol] +1 symmetric trace-free (STF) tensors of order [symbol] (with [symbol] =0 corresponding to a scalar), and also to a set of 2 [symbol] +1 spherical harmonics (SH) [symbol] (ĥ) with m=− [symbol] , . . . , [symbol] (Thorne, 1980). Hence, there is a 1-to-1 correspondence between SH and STF tensors.

All [symbol] in dMRI context are even due to time-translation invariance of the Brownian motion dictating even parity [symbol] (−\hat{n})= [symbol] (\hat{n}). Hence, each cumulant or moment tensor, as in Eq. (1), can be split into a direct sum of irreducible representations with even e, connecting it with the orientation dispersion in the SH basis (see, e.g., Novikov et al., 2018; and Pozo et al., 2019). The STF-SH equivalence can be a useful tool to construct and provide geometric meaning to the invariants.

### Exemplary Decomposition into Irreducible Representations

The tensors D, S, and A can be separated into irreducible parts, cf. Eqs. (11), (6) and (8) correspondingly. In components,

$${D}_{ij} = {D}_{00} {𝒴}_{ij}^{00} + {D}_{2 m} {𝒴}_{ij}^{2 m} , \qquad ( 17 )$$

$${S}_{ijkl} = {S}_{00} {𝒴}_{ijkl}^{00} + {S}_{2 m} {𝒴}_{ijkl}^{2 m} + {S}_{4 m} {𝒴}_{ijkl}^{4 m} ,$$

$${A}_{ij} = {A}_{00} {𝒴}_{ij}^{00} + {A}_{2 m} {𝒴}_{ij}^{2 m} .$$

Thus, [symbol] generate SH and form the standard STF tensor basis (see, e.g., Thorne, 1980). For L> [symbol] , it is possible to provide the basis tensors by symmetrization with kronecker symbols,

$${𝒴}_{{i}_{1} … {i}_{L}}^{ℓ m} = {𝒴}_{( {i}_{1} … {i}_{ℓ}}^{ℓ m} {δ}_{{i}_{ℓ + 1} {i}_{ℓ + 2}}^{} … {δ}_{{i}_{L - 1} {i}_{L )}}^{} , L \geq ℓ , \qquad ( 18 )$$

which generate standard spherical harmonics

$${Y}^{ℓ m} ( \hat{n} ) = {𝒴}_{{i}_{1} … {i}_{L}}^{ℓ m} {n}_{{i}_{1}} … {n}_{{i}_{L}} = {𝒴}_{{i}_{1} … {i}_{ℓ}}^{ℓ m} {n}_{{i}_{1}} … {n}_{{i}_{ℓ}} , L \geq ℓ . \qquad ( 19 )$$

Inverting Eq. (17), the STF components [symbol] are related to their Cartesian counterparts via

$${D}_{00} = 1 / 3 {C}_{0}^{2} {𝒴}_{ij}^{00 *} {D}_{ij} , {D}_{2 m} = 2 / 3 {C}_{2}^{2} {𝒴}_{ij}^{2 m *} {D}_{ij} , \qquad ( 20 )$$

$${S}_{00} = 1 / 5 {C}_{0}^{2} {𝒴}_{ijkl}^{00 *} {S}_{ijkl} , {S}_{2 m} = 4 / 7 {C}_{2}^{2} {𝒴}_{ijkl}^{2 m *} {S}_{ijkl} , {S}_{4 m} = 8 / 35 {C}_{4}^{2} {𝒴}_{ijkl}^{4 m *} {S}_{ijkl}$$

$${A}_{00} = 1 / 3 {C}_{0}^{2} {𝒴}_{ij}^{00 *} {A}_{ij} , {A}_{2 m} = 2 / 3 {C}_{2}^{2} {𝒴}_{ij}^{2 m *} {A}_{ij} ,$$

where

$${C}_{ℓ} = \sqrt{\frac{2 ℓ + 1}{4 π}} ,$$

and * denotes complex conjugation.

The above STF/Cartesian correspondence can be generalized to any order-L fully symmetric tensor S, which can be decomposed into a linear combination of its trace and STF tensors:

$${S}_{{i}_{1} … {i}_{L}}^{} = {\sum }_{m = - ℓ}^{ℓ} {S}_{{i}_{1} … {i}_{L}}^{( ℓ )} = {S}_{ℓ m} {𝒴}_{{i}_{1} … {i}_{L}}^{ℓ m} \qquad ( 21 )$$

where in the last equation, the sum over l and m is implied, and [symbol] = [symbol] [symbol] is the degree- [symbol] component. The general mapping from Cartesian, [symbol] , to STF basis, [symbol] , can be computed from (Thorne, 1980).

[symbol]

### 2. Rotational Invariants

Two intrinsic invariants for [symbol] =2 can be obtained from the characteristic equation

$$det ( {S}^{( 2 )} - λ \vert ) = 0 , {I}_{ij} = {δ}_{ij} , \qquad ( 24 )$$

since the coefficients of this cubic polynomial are rotationally invariant. It is also possible to express these invariants from tr (S⁽²⁾)ⁿ for n=2,3 (note that tr S⁽²⁾=0):

$$\begin{matrix}{tr ( {S}^{( 2 )} )}^{2} = \frac{3}{2} {C}_{2}^{2} {S}_{2 m} {S}_{2 m} \\ = \frac{3}{2} {C}_{2}^{2} ( {S}_{2 - 2}^{2} + {S}_{2 - 1}^{2} + {S}_{20}^{2} + {S}_{21}^{2} + {S}_{22}^{2} ) , \\ {tr ( {S}^{( 2 )} )}^{3} = \frac{9}{8} {ϵ}_{ijk} {S}_{2 m} {S}_{2 n} {S}_{2 p} {y}_{1 i}^{2 m} {y}_{2 j}^{2 n} {y}_{3 k}^{2 p} \\ = \frac{3}{8} {C}_{2}^{3} ( 3 {S}_{20} {S}_{21}^{2} + 2 {S}_{20}^{3} - 6 {S}_{20} {S}_{2 - 2}^{2} - \\ 3 \sqrt{3} {S}_{22} {S}_{2 - 1}^{2} + 6 \sqrt{3} {S}_{21} {S}_{2 - 1} {S}_{2 - 2} + \\ 3 \sqrt{3} {S}_{21}^{2} {S}_{22} + 3 {S}_{20} {S}_{2 - 1}^{2} - 6 {S}_{20} {S}_{22}^{2} )\end{matrix} , \qquad ( 25 )$$

which have a 1-to-1 mapping to the eigenvalues of S⁽²⁾

Intrinsic invariants for fourth-order tensors are more intricate since the characteristic equation can be written with more dof than in the second-order case. For the covariance or elasticity tensor this can be written as (Betten, 1987)

$$det ( C - {I}^{( 4 )} ( λ , μ ) ) = 0 , \\ {I}_{ijkl}^{( 4 )} ( λ , μ ) = λ / 2 ( {δ}_{ik} {δ}_{jl} + {δ}_{il} {δ}_{jk} ) + {μδ}_{ij} {δ}_{kl} . \qquad ( 26 )$$

To solve this problem, certain publications (see, e.g., Betten, 1987; Basser & Pajevic, 2007) and others proposed to map the elasticity fourth-order 3D tensor to a second-order 6D tensor. For C→C_{6×6}, it is also possible to use the mapping from (see, e.g., Basser & Pajevic, 2007):

$$( \begin{matrix}{C}_{1111} & {C}_{1122} & {C}_{1133} & \sqrt{2} {C}_{1112} & \sqrt{2} {C}_{1113} & \sqrt{2} {C}_{1123} \\ {C}_{1122} & {C}_{2222} & {C}_{2233} & \sqrt{2} {C}_{2212} & \sqrt{2} {C}_{2213} & \sqrt{2} {C}_{2223} \\ {C}_{1133} & {C}_{2233} & {C}_{3333} & \sqrt{2} {C}_{3312} & \sqrt{2} {C}_{3313} & \sqrt{2} {C}_{3323} \\ \sqrt{2} {C}_{1112} & \sqrt{2} {C}_{2212} & \sqrt{2} {C}_{3312} & 2 {C}_{1212} & 2 {C}_{1213} & 2 {C}_{1223} \\ \sqrt{2} {C}_{1113} & \sqrt{2} {C}_{2213} & \sqrt{2} {C}_{3313} & 2 {C}_{1312} & 2 {C}_{1313} & 2 {C}_{1323} \\ \sqrt{2} {C}_{1123} & \sqrt{2} {C}_{2223} & \sqrt{2} {C}_{3323} & 2 {C}_{2312} & 2 {C}_{2313} & 2 {C}_{2323}\end{matrix} ) . \qquad ( 27 )$$

The characteristic polynomial in Eq. (26) has degree 6 in λ and degree 3 in μ (Betten, 1987). From these coefficients, it is possible to extract invariants but these mix different irreducible representations. In the exemplary approaches utilized according to the exemplary embodiments of the present disclosure, in contrast to previous approaches, we use Eq. (26) to solve a more constrained problem: finding the intrinsic invariants of S⁽⁴⁾, which has only 9 independent parameters. One of the advantages of the exemplary approaches can be that the obtained invariants all correspond to a particular symmetry, i.e., to the irreducible representation of SO(3) group of degree [symbol] =4, which makes these invariants complementary to those coming from other irreducible representations. The corresponding characteristic polynomial is sixth-order in λ and first-order in μ. Although all these coefficients are rotationally invariant, e.g., only 4 are algebraically independent. These can be found setting μ=0 and they have a 1-to-1 mapping with the eigenvalues of S⁽⁴⁾_{6×6}, or equivalently, tr (S⁽⁴⁾)ⁿ, n=1, . . . , 6. It is noted that

$${tr ( {S}^{( 4 )} )}^{n} = {S}_{{i}_{1} {j}_{1} {i}_{2} {j}_{2}}^{( 4 )} … {S}_{{i}_{n} {j}_{n} {i}_{1} {j}_{1}}^{( 4 )} = {tr ( {S}_{6 \times 6}^{( 4 )} )}^{n} . \qquad ( 28 )$$

Initially, it is possible to think that the characteristic equation of S⁽⁴⁾ provides its 6 intrinsic invariants: tr (S⁽⁴⁾)ⁿ, n=1, . . . , 6. This is not the case. Since each tensor element satisfies the characteristic equation, it is possible to prove that tr (S⁽⁴⁾)², tr (S(4))³, tr (S⁽⁴⁾)⁴, tr (S(4))⁵ determine all traces of higher powers of S⁽⁴⁾.

The remaining 2 intrinsic invariants of S⁽⁴⁾ can be obtained from its eigentensor decomposition (see, e.g., Basser & Pajevic, 2007)

$${S}_{ijkl}^{( 4 )} = {\sum }_{a = 1}^{6} {λ}_{a} {E}_{ij}^{( a )} {E}_{kl}^{( a )} , \qquad ( 29 )$$

where λₐ and Eᵢⱼ^{(a)} are the eigenvalues and eigentensors of S⁽⁴⁾. This can be analogous to an eigenvalue decomposition of S⁽⁴⁾_{6×6} akin to Eq. (26) with μ=0. In the present case, e.g., λₐ are the eigenvalues and v^{(a)} [vₓₓ^{(a)}, v_{yy}^{(a)}, vₐₐ^{(a)}, v_{xy}^{(a)}, v_{xz}^{(a)}, v_{yz}^{(a)}] are the normalized eigenvectors, from which the following can be build

$${E}_{ij}^{( a )} = ( \begin{matrix}{v}_{xx}^{( a )} & \frac{1}{\sqrt{2}} {v}_{xy}^{( a )} & \frac{1}{\sqrt{2}} {v}_{xz}^{( a )} \\ \frac{1}{\sqrt{2}} {v}_{xy}^{( a )} & {v}_{yy}^{( a )} & \frac{1}{\sqrt{2}} {v}_{yz}^{( a )} \\ \frac{1}{\sqrt{2}} {v}_{xz}^{( a )} & \frac{1}{\sqrt{2}} {v}_{yz}^{( a )} & {v}_{zz}^{( a )}\end{matrix} ) . \qquad ( 30 )$$

Eigentensors satisfy Eᵢⱼ^{(a)}Eᵢⱼ^{(b)}=S_{ab}. From direct inspection of S⁽⁴⁾_{6×6} (as described herein) it can be seen that the eigentensor associated with a zero eigenvalue, λₐ_{₀}=0, is Eᵢⱼ^{(a}^{₀}⁾=1/\sqrt{3}δᵢⱼ, V^{(a}^{₀}⁾=1/\sqrt{3} (1, 1, 1, 0, 0, 0)^{t}. Furthermore, tr E^{(a)}=0 for all other five eigentensors a≠a₀.

To obtain the remaining two invariants of S⁽⁴⁾, the eigentensors should be utilized. The following can be defined:

$${E}_{ij} = {\sum }_{a \neq {a}_{0}} {E}_{ij}^{( a )} , and {\tilde{E}}_{ij} = {\sum }_{a} {λ}_{a} {E}_{ij}^{( a )} . \qquad ( 31 )$$

Traces of powers of E and E are rotationally invariant. As for any second-order tensor, only traces of linear, quadratic and cubic powers of these matrices are algebraically independent. Further, by construction, tr E=tr \tilde{E}=0. One can also check that tr E²=5 and tr [symbol] =tr (S⁽⁴⁾)², which is given by one of the previously found invariants. Therefore, it is possible to identify the two remaining independent intrinsic invariants of S⁽⁴⁾ with tr E³ and tr [symbol] , without the loss of generality.

Within the present approach in accordance with the exemplary embodiments of the present disclosure, it becomes straightforward to assign meaning to tr (S⁽⁴⁾)ⁿ, since these can be seen as shape tensor metrics of increasing order (n=2, . . . , 5). For tr E³ and tr [symbol] this is less intuitive. Thus, one exemplary alternative can be, e.g., to take any pair of eigentensors, say Eᵢⱼ⁽⁶⁾ and Eᵢⱼ⁽⁵⁾ (where λ₆ is the largest and λ₅ is the second largest eigenvalue), and compute the 3-dimensional rotation matrix between their bases, [symbol] _{5,6}, as shown in FIGS. 2a-2c. Such exemplary rotation matrix has three important aspects, e.g., (i) it contains independent information to that provided by the eigenvalues (although it is affected by {λₐ}); (ii) it is invariant to overall rotations of S⁽⁴⁾; (iii) even though it seems to have 3 free parameters, after fixing {λₐ}values we can observe that [symbol] _{5,6} is parametrized by two degrees of freedom φ and ψ (rotation angles). These exemplary (e.g., two) angles have a nontrivial 1-to-1 correspondence to the two invariants constructed above, tr E³ and tr [symbol] .

An alternative exemplary way to compute the main exemplary RICE invariants can be the following scalars for [symbol] =0, and the L₂-norms over m for l>0:

$$\begin{matrix}{D}_{0} = {C}_{0} {D}_{00} , & {D}_{2}^{2} = {C}_{2}^{2} {D}_{2 m} {D}_{2 m} , \\ {S}_{0} = {C}_{0} {S}_{00} , & {S}_{2}^{2} = {C}_{2}^{2} {S}_{2 m} {S}_{2 m} , \\ {A}_{0} = {C}_{0} {A}_{00} , & {A}_{2}^{2} = {C}_{2}^{2} {A}_{2 m} {A}_{2 m} .\end{matrix} {S}_{4}^{2} = {C}_{4}^{2} {S}_{4 m} {S}_{4 m} \qquad ( 32 )$$

### From Exemplary Tissue Diffusivities to D and C in Exemplary STF Basis

It is possible to present compartmental diffusion tensors as, e.g.:

$${D}_{ij}^{α} = = {D}_{00}^{α} {ij}_{00}^{} + {D}_{2 m}^{α} {ij}_{2 m}^{} , \qquad ( 33 )$$

where the STF basis allows a more intuitive separation of the degrees of freedom. The [symbol] =0 component represents the trace of the tensor (size) and the [symbol] =2 components represent the shape in a given reference frame, see FIG. 1d. Consequently, it is possible to write, e.g., all terms in D and C following Eq. (2). In such exemplary case, averages are taken over an arbitrary distribution of compartmental Dᵢⱼ^{α} without assumptions on the shape of Dᵢⱼ^{α} nor their distribution:

$${D}_{ij} = 〈 〈 {D}_{00}^{α} 〉 〉 {y}_{ij}^{00} + 〈 〈 {D}_{2 m}^{α} 〉 〉 {y}_{ij}^{2 m} , \\ {C}_{ijkl} = 〈 〈 {( {D}_{00}^{α} )}^{2} 〉 〉 {y}_{ij}^{00} {y}_{kl}^{00} + 〈 〈 {D}_{2 m}^{α} {D}_{2 {m}^{′}}^{α} 〉 〉 {y}_{ij}^{2 m} {y}_{kl}^{2 {m}^{′}} + 〈 〈 {D}_{00}^{α} {D}_{2 m}^{α} 〉 〉 ( {y}_{ij}^{00} {y}_{kl}^{2 m} + {y}_{ij}^{2 m} {y}_{kl}^{00} ) . \qquad ( 34 )$$

Using Eqs. (5), (7), and (20), it is possible to compute the STF decomposition of D, S, and A, and group them according to their degree l:

$${D}_{00} = 〈 〈 {D}_{00}^{α} 〉 〉 , \\ {S}_{00} = {C}_{0} 〈 〈 {( {D}_{00}^{α} )}^{2} 〉 〉 + \frac{{C}_{2}^{2}}{5 {C}_{0}} 〈 〈 {D}_{2 {m}^{′}}^{α} {D}_{2 {m}^{′}}^{α} 〉 〉 , \\ {A}_{00} = 2 {C}_{0} 〈 〈 {( {D}_{00}^{α} )}^{2} 〉 〉 - \frac{{C}_{2}^{2}}{2 {C}_{0}} 〈 〈 {D}_{2 {m}^{′}}^{α} , {D}_{2 {m}^{′}}^{α} 〉 〉 , \\ {D}_{2 m} = 〈 〈 {D}_{2 m}^{α} 〉 〉 , \\ {S}_{2 m} = 2 {C}_{0} 〈 〈 {D}_{00}^{α} {D}_{2 m}^{α} 〉 〉 + \frac{8}{21 {C}_{2}^{2}} 〈 〈 {D}_{2 {m}^{′}}^{α} {D}_{2 {n}^{′}}^{α} 〉 〉 {y}_{ij}^{2 {m}^{′}} {y}_{jk}^{2 {n}^{′}} {y}_{ki}^{sm} , \\ {A}_{2 m} = - 4 {C}_{0} 〈 〈 {D}_{00}^{α} {D}_{2 m}^{α} 〉 〉 + \frac{4}{3 {C}_{2}^{2}} 〈 〈 {D}_{2 {m}^{′}}^{α} {D}_{2 {n}^{′}}^{α} 〉 〉 {y}_{ij}^{2 {m}^{′}} {y}_{jk}^{2 {n}^{′}} {y}_{ki}^{2 m} , \\ {S}_{4 m} = \frac{8}{35 {C}_{4}^{2}} 〈 〈 {D}_{2 {m}^{′}}^{α} {D}_{2 {n}^{′}}^{α} 〉 〉 {y}_{( ij}^{2 {m}^{′}} {y}_{kl )}^{2 {n}^{′}} {y}_{ijkl}^{4 m} , \qquad ( 35 )$$

where [symbol] ᵢⱼ^{2m′} [symbol] ⱼₖ^{2n′} [symbol] ₖᵢ^{2m′} and [symbol] ᵢⱼ^{2m′} [symbol] ₖₗ^{2n′} [symbol] ᵢⱼₖₗ^{4m′} are proportional to the Clebsch-Gordan coefficients <2, m′, 2, n′|2, m> and <2, m′, 2, n′|4, m>, respectively. For example, the relation between the C tensor and the covariances of compartment diffusion tensors maps onto the addition of angular momenta (as discussed herein). The full exemplary (non-limiting) system can have, e.g., 27 independent linear equations with 27 unknowns, although all of them are not needed to generate some of the typical diffusion contrasts.
Conventional Contrasts from RICE

### MD, FA, MK, μFA and Diffusivity Variances

Interestingly, the first four equations in Eq. (35) are decoupled from the others. With D₀ and D₂, it is possible to compute:

$$MD = \overline{D} = \frac{1}{4 π} {\int }_{{3}^{2}} D ( \hat{n} ) d \hat{n} = \frac{1}{3} {D}_{ii} = \frac{1}{3} trD = {D}_{0} , \qquad ( 36 a )$$

$${FA}^{2} = \frac{3}{2} \frac{{V}_{λ} ( D )}{{V}_{λ} ( D ) + 3 {\overline{D}}^{2}} = \frac{3 {D}_{2}^{2}}{4 {D}_{0}^{2} + 2 {D}_{2}^{2}} , \qquad ( 36 b )$$

where V_{λ}(D)=(1/2)D₂² is the variance of the eigenvalues of D. This latter relation becomes evident if it is realized that in the eigenbasis D⁽²⁾=diag(λ₁−D̅, λ₂−D̅, λ₃−D̅). Thus, the rotational invariant

$${{D}_{2}^{2} = \frac{2}{3} tr {( {D}^{( 2 )} )}^{2} = \frac{2}{3} {\sum }_{i} ( {λ}_{i} - \overline{D} \} )}^{2} = 2 {V}_{λ} ( D )$$

(see, e.g., Novikov et al., 2018).

For mean kurtosis, it is possible to work with exemplary tensor derived metrics rather than average apparent kurtosis (see, e.g., Lu et al., 2006; Hansen et al., 2013; and Jespersen et al., 2017):

$$MK = \overline{W} = \frac{1}{4 π} {\int }_{{S}^{2}} W ( \hat{n} ) d \hat{n} = \frac{1}{5} {W}_{iijj} = \frac{1}{5} trW = {W}_{0} . \qquad ( 37 )$$

Further, it is possible to extract the variance of the eigenvalues of D^{α} averaged over the diffusion tensor distribution. For this, it is possible to repeat the process outlined above for each compartment α and then take the average:

$$\begin{matrix}〈 {V}_{λ} ( {D}^{α} ) 〉 = \frac{1}{2} 〈 {( {D}_{2}^{α} )}^{2} 〉 = \frac{1}{2} {C}_{2}^{2} 〈 {D}_{2 m}^{α} {D}_{2 m}^{α} 〉 \\ = \frac{1}{2} {C}_{2}^{2} ( 〈 〈 {D}_{2 m}^{α} {D}_{2 m}^{α} 〉 〉 + 〈 〈 {D}_{2 m}^{α} 〉 〉 〈 〈 {D}_{2 m}^{α} 〉 〉 ) \\ = \frac{10}{9} {S}_{0} - \frac{5}{9} {A}_{0} + \frac{1}{2} {D}_{2}^{2}\end{matrix} , \qquad ( 38 )$$

and use it to compute μFA (see, e.g., Westin et al., 2016; and Szczepankiewicz et al., 2016) without the need to assume axial symmetry in Dᵢⱼ^{α} or assuming a functional form for [symbol] (D):

$$\begin{matrix}μ {FA}^{2} = \frac{3}{2} \frac{〈 {V}_{λ} ( {D}^{α} ) 〉}{〈 {V}_{λ} ( {D}^{α} ) 〉 + 3 {D}^{2}} = \frac{3 〈 {( {D}_{2}^{α} )}^{2} 〉}{4 {D}_{0}^{2} + 2 〈 {( {D}_{2}^{α} )}^{2} 〉} \\ = \frac{60 {S}_{0} - 30 {A}_{0} + 27 {D}_{2}^{2}}{40 {S}_{0} - 20 {A}_{0} + 18 {D}_{2}^{2} + 36 {D}_{0}^{2}}\end{matrix} . \qquad ( 39 )$$

Inverting the complete system in Eq. (35), together with Eq. (20), facilitates a determination for the covariances of STF components starting from the conventional Cartesian ones, Eq. (2), Cᵢⱼₖₗ→ [symbol] the Cartesian expressions of C (see, e.g., Basser & Pajevic, 2007; Westin et al., 2016; and Magdoom et al., 2021), the STF basis can facilitate a deeper understanding of the rotationally invariant information. FIG. 1e shows a diagram, where 3 different types of information are immediately available: (i) size variance, contained in <<D₀₀^{α}^{²}>>, a scalar previously referred as isotropic variance VI:

$${V}_{1} = V ( {MD}^{α} ) = {C}_{0}^{2} 〈 〈 {D}_{00}^{{α}^{2}} 〉 〉 ; \qquad ( 40 )$$

(ii) shape covariances contained in [symbol] D₂ₘ^{α}D_{2m′}^{α} [symbol] , a 5×5 matrix whose trace was previously referred as anisotropic variance V_{A}:

$${V}_{A} = \frac{2}{5} 〈 {V}_{λ} ( {D}^{α} ) 〉 = \frac{{C}_{2}^{2}}{5} ( 〈 〈 {D}_{2 m}^{α} {D}_{2 m}^{α} 〉 〉 + 〈 {D}_{2 m}^{α} 〉 〈 {D}_{2 m}^{α} 〉 ) ; \qquad ( 41 )$$

(iii) size-shape covariance, contained in [symbol] D₀₀^{α}D₂ₘ^{α} [symbol] , a 5×1 vector that has been previously overlooked.

### Projecting on the Fiber Basis

Often, a main fiber population in a voxel is assumed, thus, D and W can be projected to the main axis v₁, yielding axial and radial projections:

$$AD = {D}_{\vert \vert} = D ( {v}_{1} ) , \qquad ( 42 )$$

$$RD = {D}_{⊥} = \frac{1}{2 π} {\int }_{{S}^{2}}^{} D ( \hat{n} ) δ ( \hat{n} \cdot {v}_{1} ) d \hat{n} , ,$$

$$AK = {W}_{\vert \vert} = {\overline{D}}^{2} / {D}_{\vert \vert}^{2} W ( {v}_{1} ) ,$$

$$RK = {W}_{⊥} = {\overline{D}}^{2} / {D}_{⊥}^{2} \frac{1}{2 π} {\int }_{{S}^{2}}^{} W ( \hat{n} ) δ ( \hat{n} \cdot {v}_{1} ) d \hat{n} ,$$

If D and W possess axial symmetry around this main fiber population, e.g., it is possible to compute the above maps without projecting onto v₁ and directly from RICE maps:

$${AD}_{ax , sym} = {D}_{0} + {D}_{2} , \qquad ( 43 )$$

$${RD}_{ax , sym} = {D}_{0} - ( 1 / 2 ) {D}_{2} ,$$

$${AK}_{ax , sym} = {\overline{D}}^{2} / {D}_{‖}^{2} ( {W}_{0} + {W}_{2} + {W}_{4} ) ,$$

$${RK}_{ax , sym} = {\overline{D}}^{2} / {D}_{⊥}^{2} ( {W}_{0} - ( 1 / 2 ) {W}_{2} + ( 3 / 8 ) {W}_{4} ) .$$

### Size-Shape Correlation (SSC)

According to certain exemplary embodiments of the present disclosure, all contrasts above are independent of A₂ₘ elements, which contain previously unexplored information. Following the [symbol] =2 part of the system in Eq. (35), it is possible to determine the 1×5 covariances <<D₀₀^{α}D₂ₘ^{α}>>:

$$〈 〈 {D}_{00}^{α} {D}_{2 m}^{α} 〉 〉 = \frac{7}{22 {C}_{0}} {S}_{2 m} - \frac{1}{11 {C}_{0}} {A}_{2 m} , \qquad ( 44 )$$

and define a novel invariant with size-shape correlation (SSC) information, complementary to DTI-DKI-μFA:

$$SSC = \frac{‖ 〈 〈 {D}_{00}^{α} {D}_{2 m}^{α} 〉 〉 ‖}{\sqrt{〈 〈 {D}_{00}^{{α}^{2}} 〉 〉 〈 〈 {D}_{2 m}^{α} {D}_{2 m}^{α} 〉 〉}} \qquad ( 45 )$$

This contrast contains information about the correlation of the sizes and shapes of the microscopic compartments in a voxel. The normalization is chosen akin to correlation coefficients, such that SSC∈[0, 1], where SSC=0 for independent shapes and sizes and SSC=1 for a linear relationship. Due to the norm taken over m on the numerator, SSC can only take only positive values.

### Exemplary Spherical Designs

Spherical designs (see, e.g., Seymour & Zaslavsky, 1984) are sets of N points {\hat{n}ⁱ}ᵢ₌₁^{N}∈ [symbol] ² on the unit sphere ∥\hat{n}∥=1 that for any rotation of the points scheme satisfy

$$\frac{1}{N} {\sum }_{i = 1}^{N} {f}_{L} ( {\hat{n}}^{i} ) = {\int }_{{S}^{2}}^{} {f}_{L} ( \hat{n} ) d \hat{n} = {f}_{00} / \sqrt{4 π} , \qquad ( 46 )$$

where ƒ₀₀/\sqrt{4π} is the spherical mean of ƒ_{L}(\hat{n}), which can be any function with finite degree L when expanded in spherical harmonics:

$${f}_{L} ( \hat{n} ) = {\sum }_{ℓ = 0}^{L} {\sum }_{m = - ℓ}^{ℓ} {f}_{ℓ m} {Y}^{ℓ m} ( \hat{n} ) . \qquad ( 47 )$$

The smallest spherical designs for L=2 and L=4 are provided by tetrahedron and icosahedron vertices, N=4 and N=12, respectively. For functions with ƒ_{L}(\hat{n})=ƒ_{L}(−\hat{n}), it is possible to further reduce N to half of the octahedron vertices for L=2 (the N=3 cyclic permutations of \hat{n}=(1,0,0)), and half of the icosahedron vertices for L=4 (the N=6 cyclic permutations of \hat{n}=1/\sqrt{1+φ²} (1,±φ,0), where φ=(1+\sqrt{5})/2).

the exemplary number of measurements of the minimal spherical designs can be much smaller than the total number of degrees of freedom in ƒ_{L}(\hat{n}). ƒ₄(\hat{n}) has 15 degrees of freedom but only 6 measurements suffice for an unbiased computation of their spherical average. Thus, in accordance with the exemplary embodiments of the present disclosure, it is possible to use spherical designs as a minimal way to measure the isotropic part of ƒ_{L}(\hat{n}), ƒ₀₀, since they cancel [symbol] contributions for 0< [symbol] ≤L.

### Exemplary Minimal Protocols

When all b-shells are acquired using spherical designs, it is possible to represent the dependence on Bᵢⱼ of the spherical mean signal using only the traces of the cumulant tensors: D₀₀, S₀₀, and A₀₀, Eq. (17). If the interest is only in measuring MD and MK, then two LTE shells with 4-designs (N=6) will suffice. This also provides sufficient measurements to fit D₂ₘ, elements, enabling the computation of FA. The signal expression to be fit for such a protocol would be:

$$( 48 )$$

$$log ( S ) = log ( {s}_{0} ) - {bg}_{i} {g}_{j} ( {D}_{00} {𝒴}_{ij}^{00} + {D}_{2 m} {𝒴}_{ij}^{2 m} ) + ( {b}^{2} / 6 ) {\overline{D}}^{2} {g}_{i} {g}_{j} {g}_{k} {g}_{l} {W}_{00} {𝒴}_{ijkl}^{00} ,$$

which only has 8 free parameters and can be robustly estimated from one b₀ and two N=6 distinct b-shells, totaling 12 DWI. From these, we can compute D₀, W₀, and D₂, and obtain MD, FA, and MK following Eqs. 36-37.

A similar exemplary procedure can be provided if it is desired to measure μFA. Here, a single STE measurement sensitive to [symbol] (b²) must be added to the previous protocol to provide simultaneous sensitivity to A₀₀ and insensitivity to A₂ₘ. Hence, the signal becomes:

$$log ( S ) = log ( {s}_{0} ) - {B}_{ij} ( {D}_{00} {𝒴}_{ij}^{00} + {D}_{2 m} {𝒴}_{ij}^{2 m} ) + ( 1 / 2 ) {B}_{ij} {B}_{kl} {S}_{00} {𝒴}_{ijkl}^{00} + ( 1 / 2 ) {B}_{ij} {B}_{kl} \cdot ( 1 / 6 ) ( {ϵ}_{ikp} {ϵ}_{jlp} + {ϵ}_{ilp} {ϵ}_{jkq} ) {A}_{00} {𝒴}_{pq}^{00} , \qquad ( 49 )$$

which has 9 free parameters that can be estimated from one b₀ and 13 DWI. Thus, it is possible to access D₀, D₂, S₀, and A₀, which provide us with MD, FA, MK, and μFA. Due to potential spurious time dependence of STE, e.g., 3 orthogonal rotations can be acquired.

For example, in both scenarios, it is possible to have more measurements than free parameters. However, being insensitive to high [symbol] contributions greatly reduces the number of parameters affecting the signal (which have to be estimated), thereby pushing down the limit of minimum directions needed. Here, no assumptions are made on the shapes of D, W, or C. Table 1 contrasts theoretically minimal spherical designs against previous literature and our proposed protocols.

TABLE 2
Description of four exemplary protocols (rows) acquired for each volunteer. Imaging
parameters were kept constant for all protocols and numbers denote the different directions
sampled on each shell. All b-values are in microstructure units ms/μm².
Res = 2 × 2 × 2 mm³, PF = 6/8, R_{GRAPPA} = 2, 68 slices, MB = 2, TE = 90 ms, TR = 4.2 s

| Protocol | b = 0 | b_{LTE} = 1 | 1b_{LTE} = 2 | b_{PTE} = 1.5 | b_{STE} = 1.5 | t_{acq} [min′ sec″] |
|---|---|---|---|---|---|---|
| DKI | 1 | 30 | 60 | — | — | 6′ 43″ |
| iRICE (MD + FA + MK) | 1 | 6 | 6 | — | — | 1′ 14″ |
| RICE | 2 | 30 | 60 | 30 | — | 11′ 20″ |
| iRICE (MD + | 2 | 6 | 6 | — | 3 | 1′ 56″ |
| FA + MK + μFA) | | | | | | |

### Exemplary MRI Experiments

After providing inform consent, three healthy volunteers (23 year old female, 25 year old female, 33 year old male) underwent MRI in a whole body 3T-system (e.g., Siemens Healthcare, Prisma) using a 32-channel head coil. Maxwell-compensated free gradient diffusion waveforms were used to yield linear, planar, and spherical B-tensor encoding using a prototype spin echo sequence with EPI readout (Szczepankiewicz et al., 2019). Four diffusion datasets were acquired according to Table 2. Imaging parameters: voxel size=2×2×2 mm³, T_{R}-4.2 s, T_{E}=90 ms, bandwidth=1818 Hz/Px, R_{GRAPPA}=2, partial Fourier=6/8, multiband=2. Total scan time was approximately 15 minutes per subject for all protocols.

### Exemplary Image Pre-Processing

All four protocols were processed identically and independently for each subject. Magnitude and phase data were reconstructed. Then, a phase estimation and unwinding step preceded the denoising of the complex images (see, e.g., Lemberskiy et al., 2019). Denoising was performed using the Marchenko-Pastur principal component analysis method (see, e.g., Veraart et al., 2016) on the real part of the phase-unwinded data. An advantage of denoising before taking the magnitude of the data is that Rician bias is reduced significantly. This data was also processed considering only magnitude DWI were acquired. Here, magnitude denoising and Rician bias correction were applied, these results can be found and described herein. Data was subsequently processed with the DESIGNER pipeline (see, e.g., Ades-Aron et al., 2018). Denoised images were corrected for Gibbs ringing artifacts accounting for the partial Fourier acquisition (see, e.g., Lee et al., 2021), based on re-sampling the image using local sub-voxel shifts. These images were rigidly aligned and then corrected for eddy current distortions and subject motion simultaneously (see, e.g., Smith et al., 2004). A b=0 image with reverse phase encoding was included for correction of EPI-induced distortions (see, e.g., Andersson et al., 2003). Further, DWI were locally smoothed based on similar spatial locations and signal intensities akin the method proposed by (see, e.g., Wiest-Daessle et al., 2007).

### Exemplary Parameter Estimation

Four different variants of the cumulant expansion were fit to all four datasets described in Table 2. This depended on which parameters each protocol was sensitive to. The full DKI protocol was fit with a regular DKI expression. The minimal DKI using Eq. (48). The full RICE protocol with Eq. (1), and the minimal RICE protocol with Eq. (49). Weighted linear least squares were used for fitting (see, e.g., Veraart et al., 2013). All code for RICE parameter estimation were implemented in MATLAB (R2021a, MathWorks, Natick, Massachusetts).

### Further Exemplary Spherical Designs

Exemplary spherical designs can fulfill Eq. (45). One can check that for L=2, {n}={(1,0,0), (0,1,0), (0,0,1)}satisfies

$$1 / 3 {\sum }_{i = 1}^{3} {f}_{2} ( {\hat{n}}^{i} ) = 1 / 3 {f}_{ℓ m} {𝒴}_{ij}^{ℓ m} ( {n}_{i}^{1} {n}_{j}^{1} + {n}_{i}^{2} {n}_{j}^{2} + {n}_{i}^{3} {n}_{j}^{3} ) = 1 / 3 {f}_{ℓ m} {𝒴}_{ij}^{ℓ m} ( {δ}_{11} + {δ}_{22} + {δ}_{33} ) = 1 / 3 {f}_{ℓ m} {𝒴}_{ij}^{ℓ m} {δ}_{ij} = {f}_{00} / \sqrt{4 π}$$

For L=4, {\hat{n}}=1/\sqrt{1+<φ²} {(1,φ,0), (0,1,φ), (φ,0,1), (1,−φ,0), (0,1,−φ), (−φ,0,1)}, where φ=(1+\sqrt{5})/2:

$$\begin{matrix}1 / 6 {\sum }_{i = 1}^{6} {f}_{4} ( {\hat{n}}^{( i )} ) = 1 / 6 {f}_{ℓ m} {𝒴}_{ijkl}^{ℓ m} ( {n}_{i}^{1} {n}_{j}^{1} {n}_{k}^{1} {n}_{l}^{1} + {n}_{i}^{2} {n}_{j}^{2} {n}_{k}^{2} {n}_{l}^{2} + {n}_{i}^{3} {n}_{j}^{3} {n}_{k}^{3} {n}_{l}^{3} + \\ {n}_{i}^{4} {n}_{j}^{4} {n}_{k}^{4} {n}_{l}^{4} + {n}_{i}^{5} {n}_{j}^{5} {n}_{k}^{5} {n}_{l}^{5} + {n}_{i}^{6} {n}_{j}^{6} {n}_{k}^{6} \text{?} \\ = 1 / 6 {f}_{ℓ m} {𝒴}_{ijkl}^{ℓ m} 6 / 5 ( ( {δ}_{11} {δ}_{11} + {δ}_{22} {δ}_{22} + {δ}_{33} {δ}_{33} + \\ 2 ( {δ}_{( 11} {δ}_{22 )} + {δ}_{( 11} {δ}_{33 )} + {δ}_{( 22} {δ}_{33 )} ) ) \\ = 1 / 5 {f}_{ℓ m} {𝒴}_{ijkl}^{ℓ m} {δ}_{( ij} {δ}_{kl )} = {f}_{00} / \sqrt{4 π} .\end{matrix}$$

$$\text{?} \text{indicates text missing or illegible when filed}$$

### Further Exemplary 6×6 Representation for Fully Symmetric Fourth-Order Tensors

Direct inspection of S⁽⁴⁾_{6×6} makes evident that: (i) trS⁽⁴⁾=0, (ii) One of the eigenvalues is zero (λₐ₀=0) and its associated eigenvector is v^{(a0)}=(1, 1, 1, 0, 0, 0)^{t}.

$$\text{?} = \frac{3}{\sqrt{4 π}} ( \begin{matrix}\text{?} & \text{?} & \text{?} & \text{?} & \text{?} & \text{?} \\ \text{?} & \text{?} & \text{?} & \text{?} & \text{?} & \text{?} \\ \text{?} & \text{?} & \text{?} & \text{?} & \text{?} & \text{?} \\ \text{?} & \text{?} & \text{?} & \text{?} & \text{?} & \text{?} \\ \text{?} & \text{?} & \text{?} & \text{?} & \text{?} & \text{?} \\ \text{?} & \text{?} & \text{?} & \text{?} & \text{?} & \text{?}\end{matrix} )$$

$$\text{?} \text{indicates text missing or illegible when filed}$$

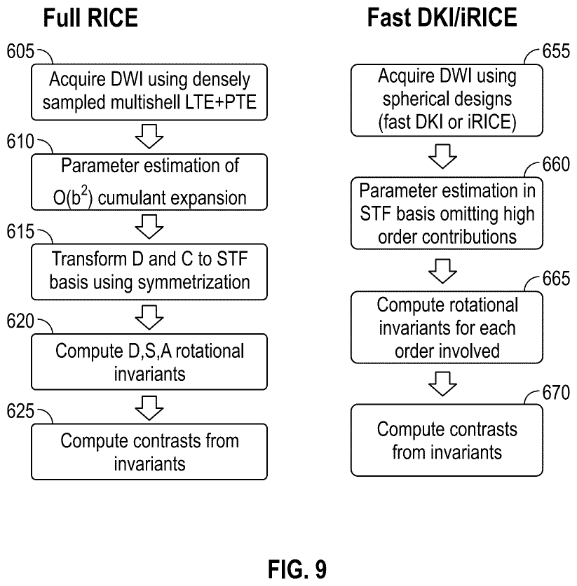

FIG. 9 shows a set of exemplary flowcharts of methods (Full RICE and FAST DKI/RICE) which can include exemplary actions which can be taken to determine exemplary tissue parameters according to exemplary embodiments of the present disclosure.

With respect to the exemplary Full RICE method, in procedure 605, it is possible to acquire DWI using densely sampled general multishell q-space trajectory acquisition (such as, but not limited to, a combination of linear tensor encoding and planar tensor encoding). Then, in procedure 610, parameter estimation of O(b²) cumulant expansion can be performed. Further, in procedure 615, D and C can be transformed to SFT basis using symmetrization. Then, in procedure 620, D, W and A rotational invariants can be computed or otherwise determined. In procedure 625, contrasts can be computed or otherwise determined from invariants.

Turning to the exemplary FAST DKI/RICE method, in procedure 655, it is possible to acquire DWI using spherical designs (e.g., fast DKI or fast RICE). Then, in procedure 660, parameter estimation in STF basis omitting high order contributions can be performed. Thereafter, in procedure 665, rotational invariants for each order involved can be computed or otherwise determined. Further, in procedure 670, contrasts can be computed or otherwise determined from invariants.

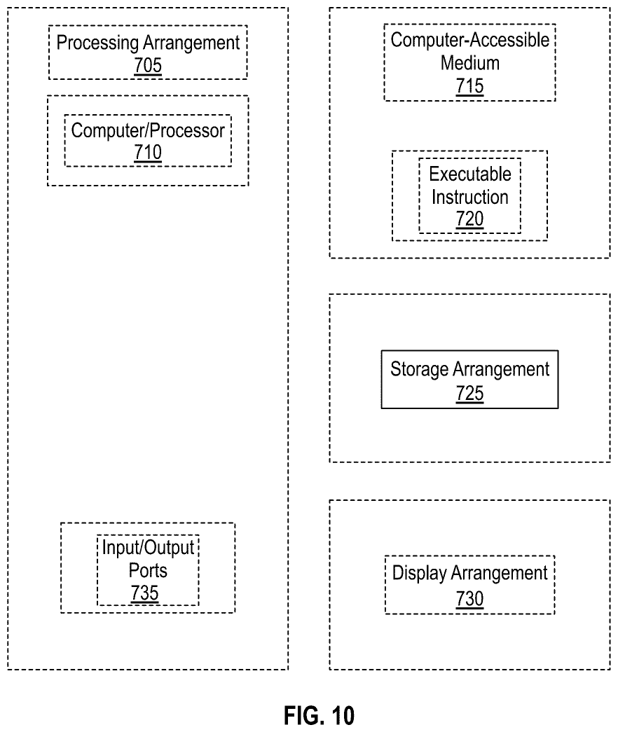

FIG. 10 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 705. Such processing/computing arrangement 705 can be, for example entirely or a part of, or include, but not limited to, a computer/processor 710 that can include, for example one or more microprocessors, and use instructions stored on a computer-accessible medium (e.g., RAM, ROM, hard drive, or other storage device).

As shown in FIG. 10, for example a computer-accessible medium 715 (e.g., as described herein above, a storage device such as a hard disk, floppy disk, memory stick, CD-ROM, RAM, ROM, etc., or a collection thereof) can be provided (e.g., in communication with the processing arrangement 705). The computer-accessible medium 715 can contain executable instructions 720 thereon. In addition or alternatively, a storage arrangement 725 can be provided separately from the computer-accessible medium 715, which can provide the instructions to the processing arrangement 705 so as to configure the processing arrangement to execute certain exemplary procedures, processes, and methods, as described herein above, for example.

Further, the exemplary processing arrangement 705 can be provided with or include an input/output ports 735, which can include, for example a wired network, a wireless network, the internet, an intranet, a data collection probe, a sensor, etc. As shown in FIG. 10, the exemplary processing arrangement 705 can be in communication with an exemplary display arrangement 730, which, according to certain exemplary embodiments of the present disclosure, can be a touch-screen configured for inputting information to the processing arrangement in addition to outputting information from the processing arrangement, for example. Further, the exemplary display arrangement 730 and/or a storage arrangement 725 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties:
1. V. G. Kiselev, Fundamentals of diffusion MRI physics, NMR in Biomedicine 30, 1 (2017).
2. D. S. Novikov, E. Fieremans, S. N. Jespersen, and V. G. Kiselev, Quantifying brain microstructure with diffusion MRI: Theory and parameter estimation, NMR in Biomedicine, e3998 (2019).
3. D. C. Alexander, T. B. Dyrby, M. Nilsson, and H. Zhang, Imaging brain microstructure with diffusion MRI: prac-ticality and applications, NMR in Biomedicine 32, e3841 (2019).
4. Y. Assaf, Can we use diffusion MRI as a bio-marker of neurodegenerative processes?, BioEssays 30, 1235 (2008).
5. I. O. Jelescu and M. D. Budde, Design and validation of diffusion MRI models of white matter, Frontiers in Physics 5, 61 (2017).
6. C.-F. Westin, H. Knutsson, O. Pasternak, F. Szczepankiewicz, E. O\ddot{z}arslan, D. van Westen, C. Mattisson, M. Bogren, L. J. O'Donnell, M. Kubicki, D. Topgaard, and M. Nilsson, q-space trajectory imaging for multidimensional diffusion MRI of the human brain, NeuroImage 135, 345 (2016).
7. D. Topgaard, Multidimensional diffusion MRI, Journal of Magnetic Resonance 275, 98 (2017).
8. N. G. van Kampen, Stochastic Processes in Physics and Chemistry, 1st ed. (Elsevier, Oxford, 1981).
9. V. G. Kiselev, The cumulant expansion: an overarch-ing mathematical framework for understanding diffusion NMR, in Diffusion MRI: Theory, Methods and Applica-tions, edited by D. K. Jones (Oxford University Press, Oxford, 2010) Chap. 10, pp. 152-168.
10. J. H. Jensen, J. A. Helpern, A. Ramani, H. Lu, and K. Kaczynski, Diffusional Kurtosis Imaging: The quan-tification of non-gaussian water diffusion by means of magnetic resonance imaging, Magnetic Resonance in Medicine 53, 1432 (2005).
11. E. O. Stejskal and T. E. Tanner, Spin diffusion measure-ments: spin echoes in the presence of a time-dependent field gradient, The Journal of Chemical Physics 42, 288 (1965).
12. K. S. Thorne, Multipole expansions of gravitational ra-diation, Rev. Mod. Phys. 52, 299 (1980).
13. L. Ning, F. Szczepankiewicz, M. Nilsson, Y. Rathi, and C.-F. Westin, Probing tissue microstructure by diffusion skewness tensor imaging, Scientific Reports 135 (2021).
14. V. G. Kiselev and K. A. Il'yasov, Is the “biexponen-tial diffusion” biexponential?, Magnetic Resonance in Medicine 57, 464 (2007).
15. S. Eriksson, S. Lasic, M. Nilsson, C.-F. Westin, and D. Topgaard, NMR diffusion-encoding with axial sym-metry and variable anisotropy: Distinguishing between prolate and oblate microscopic diffusion tensors with un-known orientation distribution, The Journal of Chemical Physics 142, 104201 (2015).
16. D. G. Cory, A. N. Garroway, and J. B. Miller, Appli-cations of spin transport as a probe of local geometry, Polymer Preprints 31, 149 (1990).
17. P. P. Mitra, Multiple wave-vector extensions of the NMR pulsed-field-gradient spin-echo diffusion measurement, Physical Review B 51, 15074 (1995).
18. S. Mori and P. C. M. Van Zijl, Diffusion weighting by the trace of the diffusion tensor within a single scan, Mag-netic Resonance in Medicine 33, 41 (1995).
19. N. Shemesh, S. N. Jespersen, D. C. Alexander, Y. Cohen, I. Drobnjac, T. B. Dyrby, J. Finterbusch, M. A. Koch, T. Kuder, F. Laun, M. Lawrenz, H. Lundell, P. P. Mitra, M. Nilsson, E. G\ddot{z}arslan, D. Topgaard, and C.-F. Westin, Conventions and nomenclature for Double Diffusion En-coding NMR and MRI, Magnetic Resonance in Medicine 75, 82 (2015).
20. G. Backus, A geometrical picture of anisotropic elastic tensors, Reviews of Geophysics 8, 633 (1970).
21. J. Betten, Irreducible invariants of fourth-order tensors, Mathematical Modelling 8, 29 (1987).
22. A. Bo'na, I. Bucataru, and M. Slawinski, Characterization of elasticity-tensor symmetries using su (2), Journal of Elasticity 75, 267 (2004).
23. M. Moakher, Fourth-order cartesian tensors: old and new facts, notions and applications, The Quarterly Journal of Mechanics and Applied Mathematics 61, 181 (2008).
24. P. J. Basser and S. Pajevic, Spectral decomposition of a 4th-order covariance tensor: Applications to diffusion tensor MRI, Signal Processing 87, 220 (2007).
25. L. Qi, D. Han, and E. X. Wu, Principal invariants and inherent parameters of diffusion kurtosis tensors, Journal of Mathematical Analysis and Applications 349, 165 (2009).
26. T. Papadopoulo, A. Ghosh, and R. Deriche, Complete set of invariants of a 4th order tensor: The 12 tasks of hardi from ternary quartics, in Medical Image Computing and Computer-Assisted Intervention—MICCAI 2014 (Springer International Publishing, Cham, 2014) pp. 233-240.
27. J. Betten, Invariants of fourth-order tensors, in Applications of tensor functions in solid mechanics, edited by J.-P. Boehler (Springer-Verlag, Vienna, Austria, 1987) Chap. 11, pp. 203-226.
28. Y. Itin and F. W. Hehl, The constitutive tensor of linear elasticity: Its decompositions, cauchy relations, null lagrangians, and wave propagation, Journal of Mathe-matical Physics 54, 042903 (2013).
29. Y. Itin and F. W. Hehl, Irreducible decompositions of the elasticity tensor under the linear and orthogonal groups and their physical consequences, in Journal of Physics: Conference Series, Vol. 597 (IOP Publishing, 2015) p. 012046.
30. A. Ghosh, T. Papadopoulo, and R. Deriche, Biomarkers for HARDI: 2nd & 4th order tensor invariants, in 2012 9th IEEE International Symposium on Biomedical Imag-ing (ISBI) (2012) pp. 26-29.
31. B. Hansen, T. E. Lund, R. Sangill, and S. N. Jespersen, Experimentally and computationally fast method for es-timation of a mean kurtosis, Magnetic Resonance in Medicine 69, 1754 (2013).
32. M. Nilsson, F. Szczepankiewicz, J. Brabec, M. Taylor, C.-F. Westin, A. Golby, D. van Westen, and P. C. Sund-gren, Tensor-valued diffusion MRI in under 3 minutes: an initial survey of microscopic anisotropy and tissue het-erogeneity in intracranial tumors, Magnetic Resonance in Medicine 83, 608 (2020).
33. M. Tinkham, Group Theory and Quantum Mechanics (Dover Books on Chemistry) (Dover Publications, 2003)
34. B. C. Hall, LieGroups, LieAlgebras, and Representations (Springer, 2015).
35. B. Hansen, T. E. Lund, R. Sangill, E. Stubbe, J. Finsterbusch, and S. N. Jespersen, Experimental considerations for fast kurtosis imaging, Magnetic resonance in medicine 76, 1455 (2016).
36. S. N. Jespersen, White matter biomarkers from diffusion MRI, Journal of Magnetic Resonance 291, 127 (2018).
37. L. Kerkelä, F. Nery, R. Callaghan, F. Zhou, N. G. Gyori, F. Szczepankiewicz, M. Palombo, G. J. Parker, H. Zhang, M. G. Hall, and C. A. Clark, Comparative analysis of sig-nal models for microscopic fractional anisotropy estima-tion using q-space trajectory encoding, NeuroImage 242, 118445 (2021).
38. F. Szczepankiewicz, C.-F. Westin, and M. Nilsson, Gradient waveform design for tensor-valued encoding in diffusion MRI, Journal of Neuroscience Methods 348, 109007 (2021).
39. A. Chakwizira, C.-F. Westin, J. Brabec, S. Lasic, L. Knutsson, F. Szczepankiewicz, and M. Nilsson, Dif-fusion MRI with pulsed and free gradient waveforms: Effects of restricted diffusion and exchange, NMR in Biomedicine 36, e4827 (2023).
40. J. Veraart, J. Sijbers, S. Sunaert, A. Leemans, and B. Jeurissen, Weighted linear least squares estimation of diffusion MRI parameters: Strengths, limitations, and pitfalls, NeuroImage 81, 335 (2013).
41. M. Herberthson, D. Boito, T. D. Haije, A. Feragen, C.-F. Westin, and E. O\ddot{z}arslan, Q-space trajectory imag-ing with positivity constraints (QTI+), NeuroImage 238, 118198 (2021).
42. R. N. Henriques, S. N. Jespersen, and N. Shemesh, Correlation tensor magnetic resonance imaging, NeuroImage 211, 116605 (2020).
43. V. G. Kiselev, Microstructure with diffusion MRI: what scale we are sensitive to?, Journal of Neuroscience Methods 347, 108910 (2021).
44. D. S. Novikov, The present and the future of microstructure MRI: From a paradigm shift to normal science, Jour-nal of Neuroscience Methods 351, 108947 (2021).
45. P. Basser and S. Pajevic, A normal distribution for tensor-valued random variables: applications to diffusion tensor MRI, IEEE Transactions on Medical Imaging 22, 785 (2003).
46. B. Jian, B. C. Vemuri, E. O\ddot{z}arslan, P. R. Carney, and T. H. Mareci, A novel tensor distribution model for the diffusion-weighted mr signal, NeuroImage 37, 164 (2007).
47. G. R. Glenn, J. A. Helpern, A. Tabesh, and J. H. Jensen, Quantitative assessment of diffusional kurtosis anisotropy, NMR in Biomedicine 28, 448 (2015).
48. S. N. Jespersen, C. D. Kroenke, L. Østergaard, J. J. H. Ackerman, and D. A. Yablonskiy, Modeling dendrite den-sity from magnetic resonance diffusion measurements, NeuroImage 34, 1473 (2007).
49. S. N. Jespersen, C. R. Bjarkam, J. R. Nyengaard, M. M. Chakravarty, B. Hansen, T. Vosegaard, L. Øster-gaard, D. Yablonskiy, N. C. Nielsen, and P. Vestergaard-Poulsen, Neurite density from magnetic resonance diffusion measurements at ultrahigh field: Comparison with light microscopy and electron microscopy, NeuroImage 49, 205 (2010).
50. E. Fieremans, J. H. Jensen, and J. A. Helpern, White matter characterization with diffusional kurtosis imag-ing, NeuroImage 58, 177 (2011).
51. H. Zhang, T. Schneider, C. A. Wheeler-Kingshott, and D. C. Alexander, NODDI: Practical in vivo neurite ori-entation dispersion and density imaging of the human brain, NeuroImage 61, 1000 (2012).
52. S. N. Sotiropoulos, T. E. Behrens, and S. Jbabdi, Ball and rackets: Inferring fiber fanning from diffusion-weighted MRI, NeuroImage 60, 1412 (2012).
53. J. H. Jensen, G. Russell Glenn, and J. A. Helpern, Fiber ball imaging, NeuroImage 124, 824 (2016).
54. D. S. Novikov, J. Veraart, I. O. Jelescu, and E. Fieremans, Rotationally-invariant mapping of scalar and ori-entational metrics of neuronal microstructure with diffu-sion MRI, NeuroImage 174, 518 (2018).
55. J. M. Pozo, S. Coelho, and A. F. Frangi, Tensorial for-mulation allowing to verify or falsify the microstructural standard model from multidimensional diffusion MRI, in Proceedings of the International Society of Magnetic Res-onance in Medicine, Vol. 3560 (Wiley, 2019).
56. H. Lu, J. Jensen, A. Ramani, and J. Helpern, Three-dimensional characterization of non-gaussian water dif-fusion in humans using diffusion kurtosis imaging, NMR in Biomedicine 19, 236 (2006).
57. S. N. Jespersen, J. L. Olesen, B. Hansen, and N. Shemesh, Diffusion time dependence of microstructural parameters in fixed spinal cord, NeuroImage 182, 329 (2017).
58. F. Szczepankiewicz, D. van Westen, E. Englund, C.-F. Westin, F. Ståhlberg, J. La'tt, P. C. Sundgren, and M. Nilsson, The link between diffusion MRI and tumor heterogeneity: Mapping cell eccentricity and density by diffusional variance decomposition (DIVIDE), NeuroIm-age 142, 522 (2016).
59. K. N. Magdoom, S. Pajevic, G. Dario, and P. J. Basser, A new framework for mr diffusion tensor distribution, Scientific Reports 11, 2766 (2021).
60. P. Seymour and T. Zaslavsky, Averaging sets: A gener-alization of mean values and spherical designs, Advances in Mathematics 52, 213 (1984).
61. F. Szczepankiewicz, C.-F. Westin, and M. Nilsson, Maxwell-compensated design of asymmetric gradient waveforms for tensor-valued diffusion encoding, Magnetic Resonance in Medicine 0, 1 (2019).
62. G. Lemberskiy, S. Baete, J. Veraart, T. Shepherd, E. Fieremans, and D. S. Novikov, Achieving sub-mm clin-ical diffusion MRI resolution by removing noise during reconstruction using random matrix theory, In Proceed-ings 27th Scientific Meeting, 0770, International Society for Magnetic Resonance in Medicine, Montreal, Canada, 2019 (2019).
63. J. Veraart, E. Fieremans, and D. S. Novikov, Diffusion MRI noise mapping using random matrix theory, Mag-netic Resonance in Medicine 76, 1582 (2016).
64. B. Ades-Aron, J. Veraart, P. Kochunov, S. McGuire, P. Sherman, E. Kellner, D. S. Novikov, and E. Fiere-mans, Evaluation of the accuracy and precision of the diffusion parameter estimation with gibbs and noise re-moval pipeline, NeuroImage 183, 532 (2018).
65. H.-H. Lee, D. S. Novikov, and E. Fieremans, Removal of partial fourier-induced gibbs (rpg) ringing artifacts in MRI, Magnetic Resonance in Medicine 86, 2733 (2021).
66. S. M. Smith, M. Jenkinson, M. W Woolrich, C. F Beck-mann, T. E J Behrens, H. Johansen-Berg, P. Bannister, M. Luca, I. Drobnjak, D. Flitney, R. Niazy, J. Saun-ders, J. Vickers, Y. Zhang, N. De Stefano, M. Brady, and P. Matthews, Advances in functional and structural mr image analysis and implementation as fsl, NeuroImage 23 Suppl 1, S208 (2004).
67. J. L. Andersson, S. Skare, and J. Ashburner, How to correct susceptibility distortions in spin-echo echo-planar images: application to diffusion tensor imaging, Neu-rolmage 20, 870 (2003).
68. N. Wiest-Daessl'e, S. Prima, P. Coup'e, S. P. Morrissey, and C. Barillot, Non-local means variants for denoising of diffusion-weighted and diffusion tensor MRI, in Med-ical Image Computing and Computer-Assisted Interven-tion (MICCAI), Vol. 10 (Springer, 2007) pp. 344-351.

1. A non-transitory computer-accessible medium having stored thereon computer-executable instructions for determining invariants associated with at least one physical structure, wherein, when a computer arrangement executes the instructions, the computer arrangement is configured to perform procedures comprising:
receiving at least one particular component which is at least one of a component of a diffusion tensor or a component of a covariance tensor, wherein the at least particular component is associated with the at least one physical structure; and
generating the invariants of at least one of the diffusion tensor or the covariance tensor based on the particular component.

2. The computer-accessible medium of claim 1, wherein at least one of
the diffusion tensor is split into a scalar part of degree 0 and a symmetric trace-free (STF) part of degree 2,
the covariance tensor is split into a fully symmetric part and an asymmetric part;
the fully symmetric part of the covariance tensor is split into a first part of degree 0, a second part of degree 2, and a third part of degree 4, or
the asymmetric part is split into a first part of degree 0 and a second part of degree 2.

3. The computer-accessible medium of claim 2, wherein at least one of:
the first parts of the diffusion tensor, the fully symmetric part of the covariance tensor and the asymmetric part of the covariance tensor, respectively, are used to generate the invariants which are proportional to full traces thereof,
the second part of the diffusion tensor is used to generate at least two of the invariants which are based on traces of second and third powers of such second part,
the second part of the fully symmetric part of the covariance tensor is used to generate at least two of the invariants which are based on traces of second and third powers of such second part,
the second part of the asymmetric part of the covariance tensor is used to generate at least two of the invariants which are based on traces of second and third powers of such second part,
the third part of the fully symmetric part of the covariance tensor is used to generate at least four of the invariants which are based on traces of second, third, fourth and fifth powers of such third part, or
the third part of the fully symmetric part of the covariance tensor is used to generate at least two invariants based on traces of cubic powers of at least two eigentensors determined from an eigentensor decomposition of such third part.

4. The computer-accessible medium of claim 2, wherein eigenbases of the second part of the fully symmetric part of the covariance tensor, the second part of the asymmetric part of the covariance tensor, and the third part of the fully symmetric part of the covariance tensor are used to generate at least one of the invariants of the covariance tensor, based on relative orientations of the eigenbases.

5. The computer-accessible medium of claim 4, wherein at least one of:
a first set of the invariants of the covariance tensor are given by parameters of a rotation of the eigenbasis of the second part of the asymmetric part of the covariance tensor relative to the eigenbasis of the second part of the fully symmetric part of the covariance tensor, or
a second set of the invariants of the covariance tensor are given by parameters of a rotation of the eigenbasis of the third part of the fully symmetric part of the covariance tensor relative to the eigenbasis of the second part of the fully symmetric part of the covariance tensor.

6. The computer-accessible medium of claim 4, wherein the eigenbasis of the third part of the fully symmetric part of the covariance tensor is generated based on the eigenbasis corresponding to a largest eigenvalue of an eigentensor decomposition thereof.

7. The computer-accessible medium of claim 3, wherein a kurtosis tensor is generated based on the fully symmetric part of the covariance tensor, and wherein kurtosis invariants are generated based on the invariants of the fully symmetric part of the covariance tensor.

8. The computer-accessible medium of claim 3, wherein the computer arrangement is configured to utilize the invariants to determine contrasts, which includes at least one of (i) mean, axial or radial diffusivity, (ii) fractional anisotropy, (iii) mean, axial and/or radial kurtosis, or (iv) microscopic fractional anisotropy.

9. The computer-accessible medium of claim 3, wherein the computer arrangement is configured to utilize the invariants to determine contrasts which includes at least one of (i) isotropic variance, or (ii) anisotropic variance.

10. The computer-accessible medium of claim 1, wherein the computer arrangement is configured to generate compartmental tensor covariances associated with tissue parameters.

11. The computer-accessible medium of claim 10, wherein the compartmental tensor covariances include size-size covariance, shape-shape covariance, and size-shape covariance.

12. The computer-accessible medium of claim 11, wherein the computer arrangement is configured to generate a size-shape correlation.

13. The computer-accessible medium of claim 1, wherein the physical structure is at least one of (i) a biological tissue, (ii) a composite material, (iii) a continuous medium, (iv) a random medium, (v) porous medium, or (vi) porous rocks.

14. The computer-accessible medium of claim 1, wherein the particular component is based on diffusion magnetic resonance (dMR) image of the at least one physical structure.

15. The computer-accessible medium of claim 1, wherein the invariants are associated with at least one parameter of at least one tissue

16. A non-transitory computer-accessible medium having stored thereon computer-executable instructions for determining invariants associated with at least one physical structure, wherein, when a computer arrangement executes the instructions, the computer arrangement is configured to perform procedures comprising:
receiving information related to at least one diffusion magnetic resonance (dMR) image of the at least one physical structure; and
generating the invariants of at least one of a diffusion tensor or covariance tensors using (i) at least one of a particular number or particular directions of diffusion acquisitions, and (ii) the information.

17-23. (canceled)

24. A non-transitory computer-accessible medium having stored thereon computer-executable instructions for determining at least one component of at least one tensor associated with at least one physical structure, wherein, when a computer arrangement executes the instructions, the computer arrangement is configured to perform procedures comprising:
receiving first information related to at least one diffusion magnetic resonance (dMR) image of the at least one physical structure;
receiving second information related to at least one constraint on the at least one component of the at least one tensor; and
generating the at least one component which is at least one of a component of a diffusion tensor and a component of a covariance tensor based on the first information and the second information.

25-30. (canceled)

31. A method for determining invariants associated with at least one physical structure, comprising:
receiving at least one particular component which is at least one of a component of a diffusion tensor or a component of a covariance tensor, wherein the at least particular component is associated with the at least one physical structure; and
generating the invariants of at least one of the diffusion tensor or the covariance tensor based on the particular component.

32-45. (canceled)

46. A method for determining invariants associated with at least one physical structure, comprising:
receiving information related to at least one diffusion magnetic resonance (dMR) image of the at least one physical structure; and
generating the invariants of at least one of a diffusion tensor or covariance tensors using (i) at least one of a particular number or particular directions of diffusion acquisitions, and (ii) the information.

47-53. (canceled)

54. A method for determining at least one component of at least one tensor associated with at least one physical structure, comprising:
receiving first information related to at least one diffusion magnetic resonance (dMR) image of the at least one physical structure;
receiving second information related to at least one constraint on the at least one component of the at least one tensor; and
generating the at least one component which is at least one of a component of a diffusion tensor and a component of a covariance tensor based on the first information and the second information.

55-90. (canceled)
